(19) United States
(12) Patent Application Publication
(10) Pub. No.: US 2006/0262896 A1
(43) Pub. Date: Nov. 23, 2006

(54) Scan parameter setting method for shuttle mode helical scan and X-ray CT apparatus

(75) Inventors: Akihiko Nishide, Tokyo (JP); Akie Katsuki, Tokyo (JP); Masae Shinya, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/435,915

(22) Filed: May 17, 2006

(30) Foreign Application Priority Data
May 19, 2005 (JP) 2005-146355

(51) Int. Cl. A61B 6/00; G01N 23/00; G21K 1/12; H05G 1/60

(52) U.S. Cl. 378/15

(57) ABSTRACT

A scout image of a subject is displayed. An operator designates at least one range in a body-axis direction, of the scout image. Further, the operator graphically inputs or key-inputs and sets imaging condition parameters such as a helical pitch, a noise index and the like for a shuttle mode helical scan with being made independent every ranges. Thus, conditions such as helical pitch and noise index for the shuttle mode helical scan can efficiently and intelligibly be set independently for each region or organ, and hence the control/optimization of imaging conditions is enabled.

### BACKGROUND OF THE INVENTION

The present invention relates to a scan parameter setting method for a shuttle mode helical scan and an X-ray CT (Computed Tomography) apparatus, and more specifically to a scan parameter setting method for a shuttle mode helical scan and an X-ray CT apparatus, which are capable of efficiently and intelligibly setting parameters for imaging conditions, such as a helical pitch for the shuttle mode helical scan, a noise index corresponding to an index of image noise of each tomographic image, etc. and reducing exposure to a patient while image quality is being held.

There has heretofore been known an X-ray CT apparatus wherein when one reconstruction area is designated based on a scout image (scanogram image) after the completion of input of scan parameters such as a slice thickness, a helical pitch, a tube voltage, a tube current, etc., a scan area or field is calculated from the reconstruction area, and the calculated scan field is helically scanned using the previously inputted scan parameters (refer to, for example, a patent document 1).

[Patent Document 1] Japanese Unexamined Patent Publication No. Hei 11(1999)-146871([0059] [0069] [0062])

The conventional X-ray CT apparatus is accompanied by a problem that the setting of scan parameters for such a shuttle mode helical scan that one-direction scan or a shuttle scan is repeated with respect to a predetermined range has not been taken into consideration.

### SUMMARY OF THE INVENTION

Therefore, an object of the present invention is to provide a scan parameter setting method for a shuttle mode helical scan and an X-ray CT apparatus, which are capable of efficiently and intelligibly setting parameters for imaging conditions, such as a helical pitch and a noise index for the shuttle mode helical scan, etc. and reducing exposure to a patient while image quality is being held.

In a first aspect, the present invention provides a scan parameter setting method for a shuttle mode helical scan, comprising the steps of displaying a scout image of a subject, allowing an operator to designate at least one range in a body-axis direction, of the scout image, and allowing the operator to graphically input or key-input and set a helical pitch for the shuttle mode helical scan in association with the range.

In the scan parameter setting method for the shuttle mode helical scan, according to the first aspect, the operator designates a range for each region or organ by referring to the displayed scout image. Then, the operator sets a helical pitch for the shuttle mode helical scan with respect to the designated range. Thus, the helical pitch for the shuttle mode helical scan can efficiently and intelligibly be set independently for each region or organ, and hence the control/optimization of imaging conditions is enabled.

In a second aspect, the present invention provides a scan parameter setting method for a shuttle mode helical scan, comprising the steps of displaying a scout image of a subject, allowing an operator to designate at least one range in a body-axis direction, of the scout image, and allowing the operator to graphically input or key-input and set a noise index for the shuttle mode helical scan in association with the range.

In the scan parameter setting method for the shuttle mode helical scan, according to the second aspect, the operator designates a range for each region or organ by referring to the displayed scout image. Then, the operator sets a noise index for the shuttle mode helical scan with respect to the designated range. Thus, the noise index for the shuttle mode helical scan can efficiently and intelligibly be set independently for each region or organ, and hence the control/optimization of imaging conditions is enabled. Since a target value of image noise of each tomographic image is fixed, the application of X rays can be controlled for each tomographic image and a reduction in exposure can also be realized.

In a third aspect, the present invention provides a scan parameter setting method for a shuttle mode helical scan, according to the first aspect, further including a step for allowing the operator to graphically input or key-input and set a noise index for the shuttle mode helical scan in association with the range.

In the scan parameter setting method for the shuttle mode helical scan, according to the third aspect, the operator designates a range for each region or organ by referring to the displayed scout image. Then, the operator sets a helical pitch and a noise index for the shuttle mode helical scan with respect to the designated range. Thus, in addition to the first aspect, the helical pitch and the noise index for the shuttle mode helical scan can efficiently and intelligibly be set independently for each region or organ, and hence the control/optimization of imaging conditions is enabled.

In a fourth aspect, the present invention provides a scan parameter setting method for a shuttle mode helical scan, according to each of the first to third aspects, further including a step for allowing the operator to graphically input or key-input and set at least one of a tube voltage and a tube current for the shuttle mode helical scan in association with the range.

In the scan parameter setting method for the shuttle mode helical scan, according to the fourth aspect, the operator designates a range for each region or organ by referring to the displayed scout image. Then, the operator sets the helical pitch and/or the noise index with respect to the designated range and sets at least one of the tube voltage and the tube current. Thus, in addition to the first through third aspects, at least one of the tube voltage and the tube current for the shutter mode helical scan can efficiently and intelligibly be set independently for each region or organ and hence the control/optimization of imaging conditions is enabled.

In a fifth aspect, the present invention provides a scan parameter setting method for a shuttle mode helical scan, having such a constitution as described above, which further includes a step for allowing the operator to set at least one of a slice thickness, a detector row number, a table speed, the number of tomographic images, a tomographic image interval and table acceleration for the shuttle mode helical scan in association with the range.

In the scan parameter setting method for the shuttle mode helical scan, according to the fifth aspect, at least one of the slice thickness, detector row number, table speed, the number of tomographic images, tomographic image interval and table acceleration for the shuttle mode helical scan can be set independently for each region or organ in addition to the first through fourth aspects, and hence the control/optimization of imaging conditions is enabled.

In a sixth aspect, the present invention provides a scan parameter setting method for a shuttle mode helical scan, according to the first aspect, further including a step for defining the one range as one group and setting one series comprising one or more groups.

In the scan parameter setting method for the shuttle mode helical scan, according to the sixth aspect, the operator sets one series (one in which groups are linked) comprising one or more groups with the designated one range as a group (parameter group associated with one range). Thus, in addition to the first aspect, helical pitches for the shuttle mode helical scan can collectively be managed with respect to a plurality of regions or organs and the control/optimization of imaging conditions is enabled.

In a seventh aspect, the present invention provides a scan parameter setting method for a shuttle mode helical scan, according to the second aspect, further including a step for defining the one range as one group and setting one series comprising one or more groups.

In the scan parameter setting method for the shuttle mode helical scan, according to the seventh aspect, the operator sets one series (one in which groups are linked) comprising one or more groups with the designated one range as a group (parameter group associated with one range). Thus, in addition to the first aspect, noise indexes for the shuttle mode helical scan can collectively be managed with respect to a plurality of regions or organs and the control/optimization of imaging conditions is enabled.

In an eight aspect, the present invention provides a scan parameter setting method for a shuttle mode helical scan, according to the third aspect, further including a step for defining the one range as one group and setting one series comprising one or more groups.

In the scan parameter setting method for the shuttle mode helical scan, according to the eighth aspect, the operator sets one series (one in which groups are linked) comprising one or more groups with the designated one range as a group (parameter group associated with one range). Thus, in addition to the third aspect, a helical pitch and a noise index for the shuttle mode helical scan can collectively be managed with respect to a plurality of regions or organs and the control/optimization of imaging conditions is made possible.

In a ninth aspect, the present invention provides a scan parameter setting method for a shuttle mode helical scan, according to the fourth aspect, further including a step for defining the one range as one group and setting one series comprising one or more groups.

In the scan parameter setting method for the shuttle mode helical scan, according to the ninth aspect, the operator sets one series (one in which groups are linked) comprising one or more groups with the designated one range as a group (parameter group associated with one range). Thus, in addition to the fourth aspect, a tube current and a tube voltage for the shuttle mode helical scan can collectively be managed with respect to a plurality of regions or organs and the control/optimization of imaging conditions is enabled.

In a tenth aspect, the present invention provides a scan parameter setting method for a shuttle mode helical scan, according to each of the first through ninth aspects, wherein the one range is set corresponding to one organ or region.

In the scan parameter setting method for the shuttle mode helical scan, according to the tenth aspect, the operator sets one organ or region as one range. Thus, the control/optimization of a helical pitch, a noise index, a tube current and a tube voltage for the shuttle mode helical scan can be carried out in organ units or region units.

In an eleventh aspect, the present invention provides a scan parameter setting method for a shuttle mode helical scan, according to each of the first through tenth aspects, wherein a default value of at least one scan parameter or a previous set value is automatically set as a candidate for a set value with respect to the designated one range.

The scan parameter setting method for the shuttle mode helical scan, according to the eleventh aspect has an advantage that since the default value or previous set value corresponding to one range is automatically set as the candidate for the set value when the operator designates the one range, time and efforts for their settings can be saved where the default value or the previous set value is used as it is.

In a twelfth aspect, the present invention provides a scan parameter setting method for a shuttle mode helical scan, according to each of the first through eleventh aspects, wherein the X-ray CT scan is of a shuttle mode variable pitch helical scan or a shuttle mode variable speed helical scan for collecting data even at the time of a start of linear movement and an end thereof, and even in acceleration or deceleration at midstream thereof.

The scan parameter setting method for the shuttle mode helical scan, according to the twelfth aspect can obtain an effect according to each of the first through eleventh aspects even in the case of the shuttle mode variable pitch helical scan or the shuttle mode variable speed helical scan.

In a thirteenth aspect, the present invention provides an X-ray CT apparatus comprising an X-ray tube, a detector, helical scan means for rotating at least one of the X-ray tube and the detector about a target to be imaged and collecting data while both are being moved linearly relative to the target to be imaged, scan parameter setting means for allowing an operator to set scan parameters for a helical scan, and image reconstructing means for reconstructing an image, based on the collected data, wherein the scan parameter setting means displays a scout image of a subject and when the operator designates at least one range in a body-axis direction, of the scout image and graphically inputs or key-inputs a helical pitch for the shuttle mode helical scan in association with the range, the scan parameter setting means sets the inputted helical pitch as a scan parameter for the shuttle mode helical scan, corresponding to the range.

The X-ray CT apparatus according to the thirteenth aspect is capable of suitably executing the scan parameter setting method for the shuttle mode helical scan, according to the first aspect.

In a fourteenth aspect, the present invention provides an X-ray CT apparatus comprising an X-ray tube, a detector, helical scan means for rotating at least one of the X-ray tube and the detector about a target to be imaged and collecting data while both are being moved linearly relative to the target to be imaged, scan parameter setting means for allowing an operator to set parameters for a helical scan, and image reconstructing means for reconstructing an image, based on the collected data, wherein the scan parameter setting means displays a scout image of a subject and when the operator designates at least one range in a body-axis direction, of the scout image and graphically inputs or key-inputs a noise index for the shuttle mode helical scan in association with the range, the scan parameter setting means sets the inputted noise index as a scan parameter for the shuttle mode helical scan, corresponding to the range.

The X-ray CT apparatus according to the fourteenth aspect is capable of suitably implementing the scan parameter setting method for the shuttle mode helical scan, according to the second aspect.

In a fifteenth aspect, the present invention provides an X-ray CT apparatus according to the thirteenth aspect, wherein when the operator graphically inputs or key-inputs a noise index for the shuttle mode helical scan in association with the range, the parameter setting means sets the inputted noise index as a scan parameter for the shuttle mode helical scan, corresponding to the range.

The X-ray CT apparatus according to the fifteenth aspect is capable of suitably executing the scan parameter setting method for the shuttle mode helical scan, according to the third aspect.

In a sixteenth aspect, the present invention provides an X-ray CT apparatus according to each of the thirteenth through fifteenth aspects, wherein when the operator graphically inputs or key-inputs at least one of a tube voltage and a tube current for the shuttle mode helical scan in association with the range, the parameter setting means sets at least one thereof as a scan parameter for the shuttle mode helical scan, corresponding to the range.

The X-ray CT apparatus according to the sixteenth aspect is capable of suitably executing the scan parameter setting method for the shuttle mode helical scan, according to the fourth aspect.

In a seventeenth aspect, the present invention provides an X-ray CT apparatus having above constitution, wherein when the operator inputs at least one of a slice thickness, a detector row number, a table speed, the number of tomographic images, a tomographic image interval, and table acceleration for the shuttle mode helical scan in association with each range referred to above, the parameter setting means sets at least one thereof as a scan parameter for the shuttle mode helical scan, corresponding to the range.

The X-ray CT apparatus according to the seventeenth aspect is capable of suitably executing the scan parameter setting method for the shuttle mode helical scan, according to the fifth aspect.

In an eighteenth aspect, the present invention provides an X-ray CT apparatus according to the thirteenth aspect, wherein the parameter setting means is capable of setting one series comprising one or more groups with the above one range as one group, and when execution of one series is instructed, the helical scan means continuously executes a shuttle mode helical scan for a group which belongs to the corresponding series.

The X-ray CT apparatus according to the eighteenth aspect is capable of suitably executing the scan parameter setting method for the shuttle mode helical scan, according to the sixth aspect.

In a nineteenth aspect, the present invention provides an X-ray CT apparatus according to the fourteenth aspect, wherein the parameter setting means is capable of setting one series comprising one or more groups with the above one range as one group, and when execution of one series is instructed, the helical scan means continuously executes a shuttle mode helical scan for a group which belongs to the corresponding series.

The X-ray CT apparatus according to the nineteenth aspect is capable of suitably executing the scan parameter setting method for the shuttle mode helical scan, according to the seventh aspect.

In a twentieth aspect, the present invention provides an X-ray CT apparatus according to the fifteenth aspect, wherein the parameter setting means is capable of setting one series comprising one or more groups with the above one range as one group, and when execution of one series is instructed, the helical scan means continuously executes a shuttle mode helical scan for a group which belongs to the corresponding series.

The X-ray CT apparatus according to the twentieth aspect is capable of suitably executing the scan parameter setting method for the shuttle mode helical scan, according to the eighth aspect.

In a twenty-first aspect, the present invention provides an X-ray CT apparatus according to the sixteenth aspect, wherein the parameter setting means is capable of setting one series comprising one or more groups with the above one range as one group, and when execution of one series is instructed, the helical scan means continuously executes a shuttle mode helical scan for a group which belongs to the corresponding series.

The X-ray CT apparatus according to the twenty-first aspect can suitably execute the scan parameter setting method for the shuttle mode helical scan, according to the ninth aspect.

In a twenty-second aspect, the present invention provides an X-ray CT apparatus according to each of the thirteenth through twenty-first aspects, wherein the parameter setting means sets the above one range in association with one organ or region.

The X-ray CT apparatus according to the twenty-second aspect is capable of suitably executing the scan parameter setting method for the shuttle mode helical scan, according to the tenth aspect.

In a twenty-third aspect, the present invention provides an X-ray CT apparatus according to each of the thirteenth through twenty-second aspects, wherein the parameter setting means automatically sets a default value of at least one scan parameter or a previous set value as a candidate for a set value with respect to the designated one range.

The X-ray CT apparatus according to the twenty-third aspect is capable of suitably executing the scan parameter setting method for the shuttle mode helical scan, according to the eleventh aspect.

In a twenty-fourth aspect, the present invention provides an X-ray CT apparatus according to each of the thirteenth through twenty-third aspects, wherein the helical scan means performs a shuttle mode variable pitch helical scan or a shuttle mode variable speed helical scan for collecting data even at the time of a start of linear movement thereof and an end thereof and even in acceleration or deceleration at midstream thereof.

The X-ray CT apparatus according to the twenty-fourth aspect is capable of suitably executing the scan parameter setting method fort the shuttle mode helical scan, according to the twelfth aspect.

According to a scan parameter setting method for a shuttle mode helical scan, and an X-ray CT apparatus according to the present invention, which are capable of efficiently and intelligibly setting parameters for imaging conditions, such as a helical pitch and a noise index for the shuttle mode helical scan, etc. independently every regions or organs.

The scan parameter setting method for the shuttle mode helical scan and the X-ray CT apparatus according to the present invention can be used in a medical site.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an X-ray CT apparatus according to an embodiment 1.

FIG. 2 is an explanatory diagram illustrating rotation of an X-ray tube and a multi-row X-ray detector.

FIG. 3 is a flowchart depicting a schematic operation of the X-ray CT apparatus according to the embodiment 1.

FIG. 4 is a flowchart showing the details of a helical scan parameter setting process.

FIG. 5 is a flowchart following FIG. 4.

FIG. 6 is a first illustrative diagram of a scan parameter setting screen.

FIG. 7 is a diagram illustrating a patient information screen and a protocol selection screen.

FIG. 8 is a first illustrative diagram of a protocol list screen.

FIG. 9 is a second illustrative diagram of a protocol list screen.

FIG. 10 is a diagram illustrating a scan type setting screen.

FIG. 11 is a diagram illustrating a scan parameter selection screen.

FIG. 12 is a diagram illustrating a scout scan screen.

FIG. 13 is a first illustrative diagram of a scout image display screen.

FIG. 14 is a second illustrative diagram of a scan parameter setting screen.

FIG. 15 is a second illustrative diagram of a scout image display screen.

FIG. 16 is a third illustrative diagram of a scan parameter setting screen.

FIG. 17 is a diagram illustrating a noise index setting screen.

FIG. 18 is a diagram illustrating a slice thickness/and the like setting screen.

FIG. 19 is a fourth illustrative diagram of a scan parameter setting screen.

FIG. 20 is a diagram illustrating a tube current/and the like display screen.

FIG. 21 is a diagram illustrating a slice thickness/and the like display screen.

FIG. 22 is a fifth illustrative diagram of a scan parameter setting screen.

FIG. 23 is a diagram illustrating a screen indicative of changes in main scan parameters using a scout image.

FIG. 24 is an explanatory diagram showing changes in table speed.

FIG. 25 is a diagram illustrating a series registration screen.

FIG. 26 is a first illustrative diagram of a scan progress screen.

FIG. 27 is a second illustrative diagram of a scan progress screen.

FIG. 28 is a flowchart showing the details of a data acquisition process (process 2 of FIG. 3).

FIG. 29 is a flowchart showing the details of an image reconstructing process (process 3 of FIG. 3).

FIG. 30 is an explanatory diagram showing rowwise filter coefficients.

FIG. 31 is an explanatory diagram showing a slice thick in slice thickness at the periphery of a reconstruction area as compared with its center.

FIG. 32 is an explanatory diagram illustrating rowwise filter coefficients different according to channels.

FIG. 33 is an explanatory diagram showing a slice uniform in slice thickness even at both the center and periphery of a reconstruction area.

FIG. 34 is an explanatory diagram showing rowwise filter coefficients for thinning a slice thickness.

FIG. 35 is flowchart showing the details of a three-dimensional image reconstructing process.

FIG. 36 is a conceptual diagram showing a state in which pixel rows on a reconstruction area P are projected in an X-ray penetration direction.

FIG. 37 is a conceptual diagram illustrating lines formed by projecting the pixel rows on the reconstruction area P onto a detector surface or plane.

FIG. 38 is a conceptual diagram showing a state in which projection data Dr at a view angle view=0° is projected on the reconstruction area P.

FIG. 39 is a conceptual diagram depicting backprojection pixel data D2 on the reconstruction area P at the view angle view=0°.

FIG. 40 is an explanatory diagram showing a state in which backprojection pixel data D2 are added over all views in association with pixels to obtain backprojection data D3.

FIG. 41 is a conceptual diagram illustrating a circular reconstruction area R.

### DETAILED DESCRIPTION OF THE INVENTION

The present invention will hereinafter be explained in further detail according to embodiments illustrated in the accompanying drawings. Incidentally, the present invention is not limited to or by the embodiments.

### EMBODIMENT 1

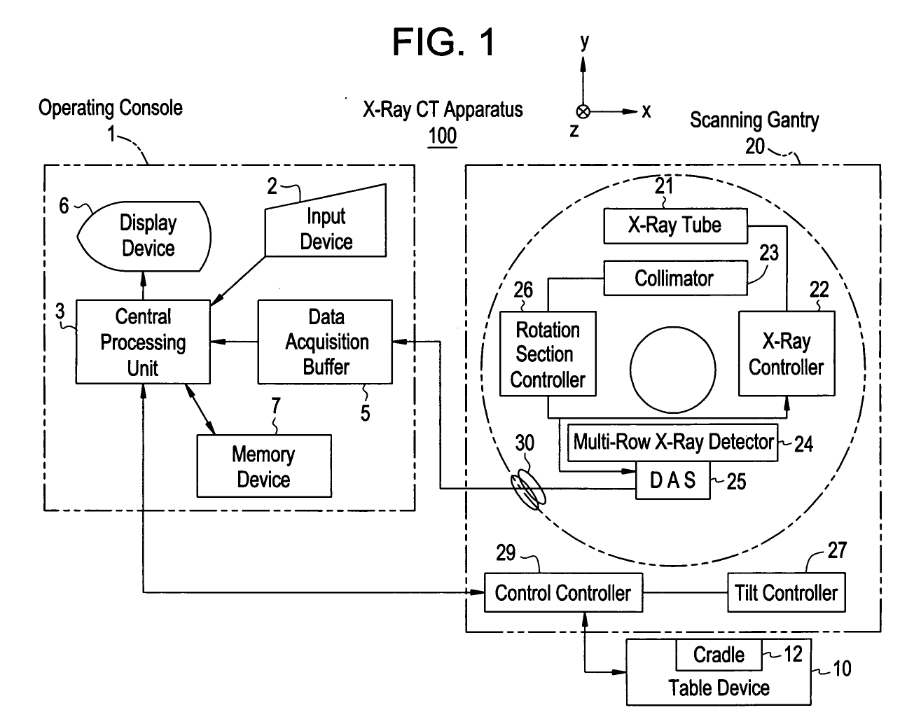

FIG. 1 is a configurational block diagram showing an X-ray C apparatus 100 according to an embodiment 1.

The present X-ray CT apparatus 100 is equipped with an operating console 1, a table device 10 and a scanning gantry 20.

The operating console 1 includes an input device 2 which receives an operator's input, a central processing unit 3 which executes an image reconstructing process, etc., a data acquisition buffer 5 which collects projection data obtained by the scanning gantry 20, a display device 6 which displays a tomographic image reconstructed from the projection data, and a memory device 7 which stores programs, data and an X-ray tomographic image therein. Incidentally, the display device 6 is configured as a multiscreen display having two screens of a right screen and a left screen.

The table device 10 includes a cradle 12 which places a subject thereon and inserts and draws it into and from a bore (cavity section). The cradle 12 is elevated (in a y-axis direction) and linearly moved (in a z-axis direction) by a motor built in the table device 10.

The scanning gantry 20 is provided with an X-ray tube 21, an X-ray controller 22, a collimator 23, a multi-row X-ray detector 24, a DAS (Data Acquisition System) 25, a rotation section controller 26 which rotates the X-ray tube 21 or the like about a body axis of the subject, a tilt controller 27 which performs control of the scanning gantry 20 at the time that the scanning gantry 20 is tilted forward or backward of its rotational axis, a control controller 29 which performs a transfer of a control signal or the like between the operating console 1 and the bed or table device 10, and a slip ring 30.

The amount of linear movement of the cradle 12 is counted by an encoder built in the table device 10. The control controller 29 calculates a z-axis coordinate of the cradle 12 from the amount of linear movement and sends it to the DAS 25 through the slip ring 30.

The DAS 25 AD-converts projection data acquired by the multi-row X-ray detector 24 and adds the z-axis coordinate thereto. Then, the DAS 25 transfers the same data to the data acquisition buffer 5 through the slip ring 30.

The central processing unit 3 effects a pre-process and an image reconstructing process on the projection data collected by the data acquisition buffer 5 to generate a tomographic image, which in turn is displayed on the display device 6.

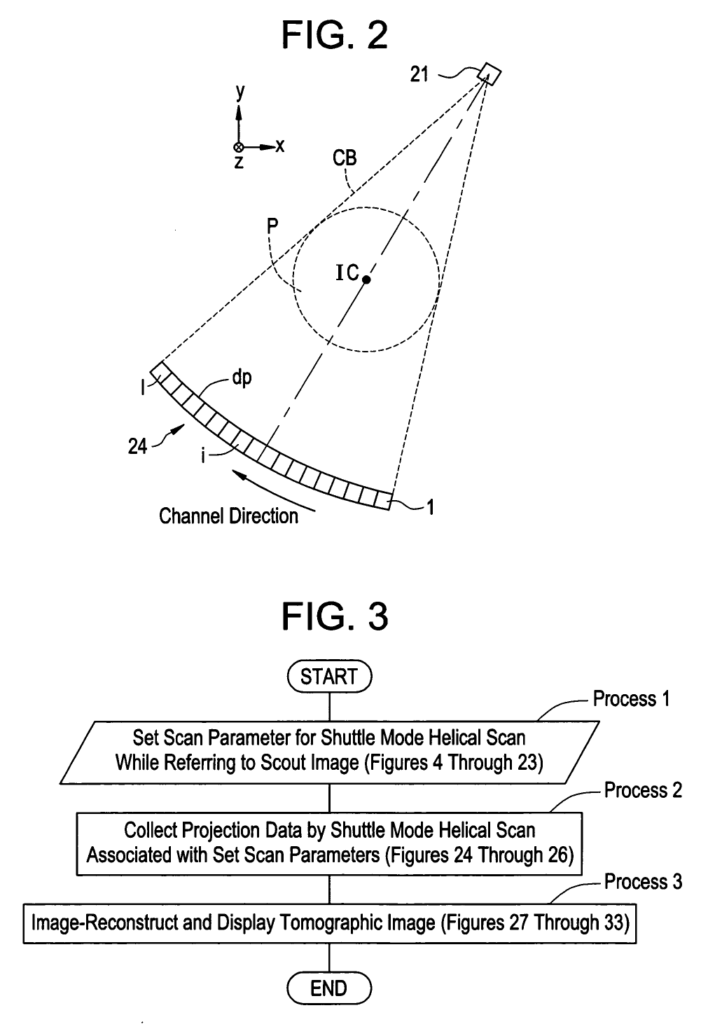

FIG. 2 is an explanatory diagram of the X-ray tube 21 and the multi-row X-ray detector 24.

The X-ray tube 21 and the multi-row X-ray detector 24 are rotated about the center of rotation IC. The vertical direction is defined as a y direction, the moving or traveling direction of the cradle 12 is defined as a z direction, and the direction orthogonal to the y direction and the z direction is defined as an x direction. When they are not tilted, the plane on which the X-ray tube 21 and the multi-row X-ray detector 24 are rotated, is an xy plane.

The X-ray tube 21 generates an x-ray beam called “X-ray cone beam CB”. When the direction of a central axis of the X-ray cone beam CB is parallel to the y direction, a view angle is assumed to be 0°.

The multi-row X-ray detector 24 has detector rows corresponding to, for example, 64 rows. The respective detector rows have channels equivalent to, for example, 1024 channels.

FIG. 3 is a flowchart showing the outline of operation of the X-ray CT apparatus 100.

In a process 1, a scout image of a subject is photographed or imaged and displayed. An operator designates one or more ranges in a body-axis direction, of the scout image. Further, the operator graphically inputs or key-inputs and sets scan parameters for a shuttle mode helical scan, such as a helical pitch, a noise index, etc. in association with the ranges. This process 1 will be described in detail later.

In a process 2, projection data are collected by the shuttle mode helical scan associated with the set scan parameters. This process 2 will be explained in detail later.

In a process 3, a tomographic image is image-reconstructed from the collected projection data and the so-processed tomographic image is displayed on the display device 6. This process 3 will be explained in detail later.

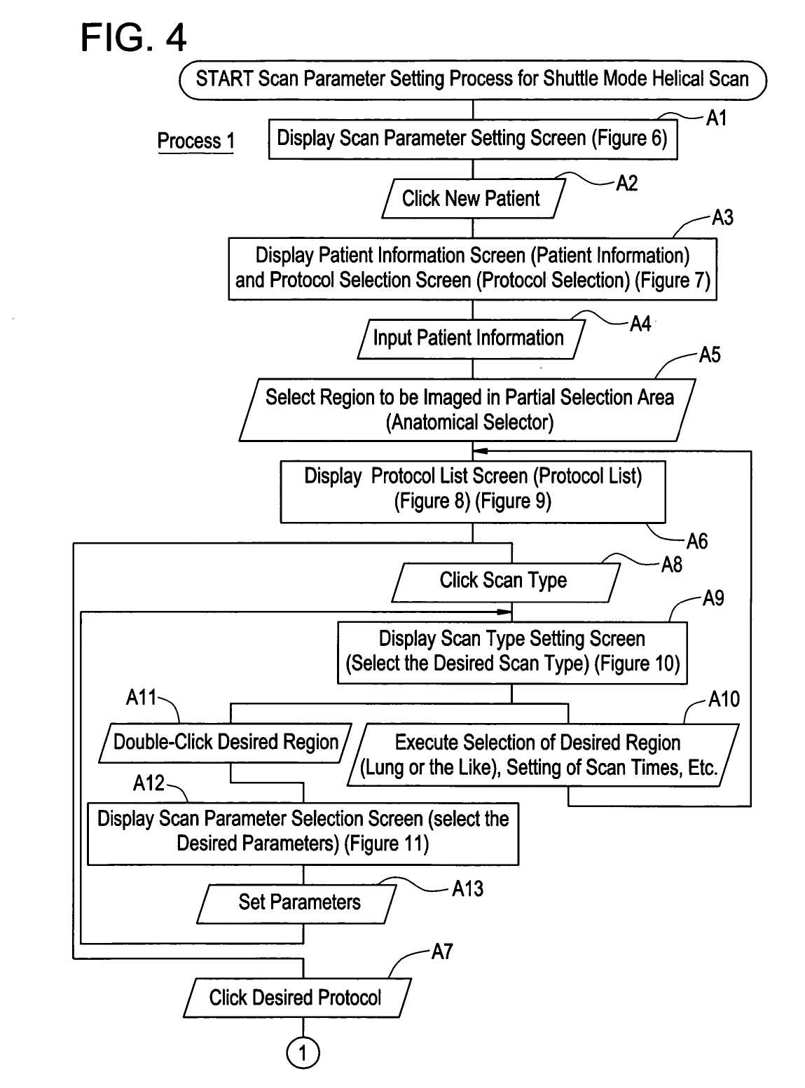

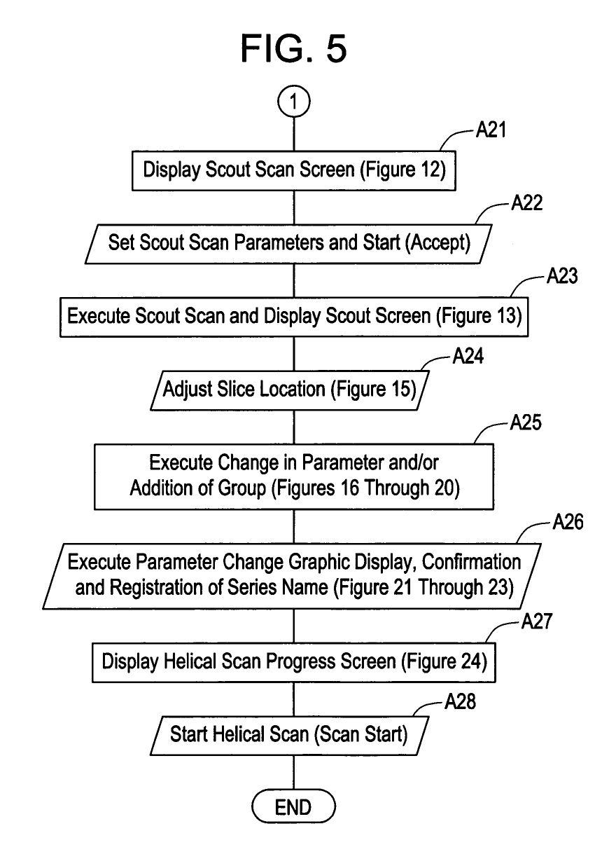

FIGS. 4 and 5 are flowcharts showing the details of processing for setting the helical scan parameters for the shuttle mode helical scan (process 1).

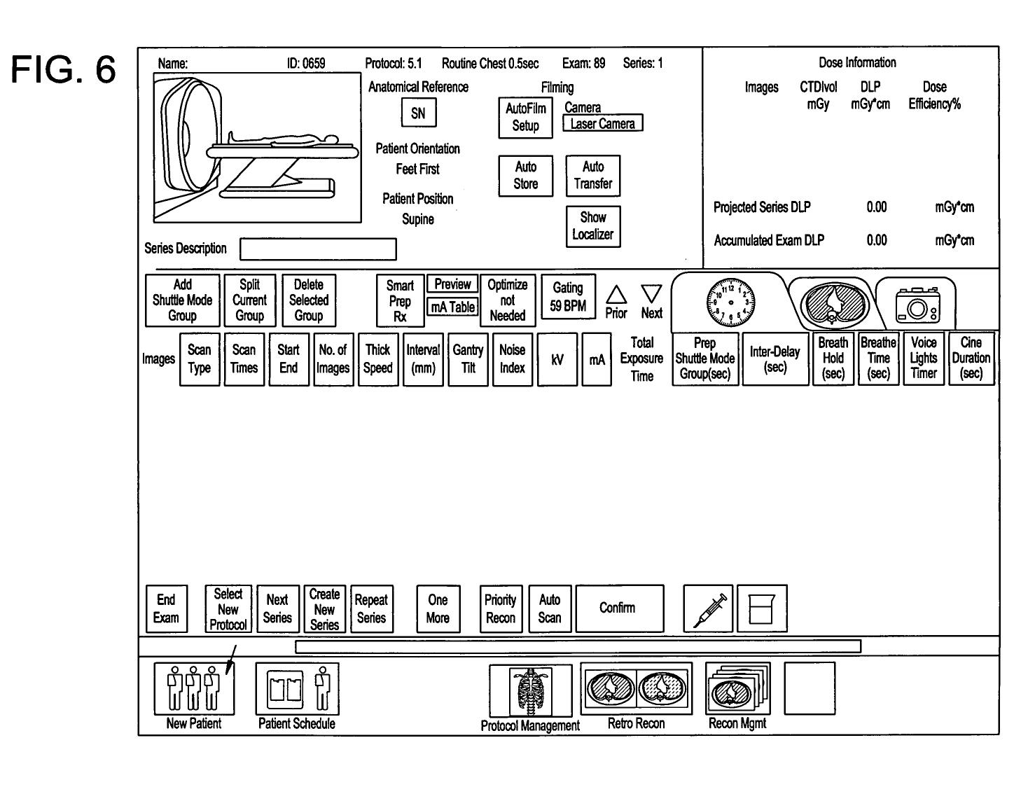

At Step A1 of FIG. 4, the central processing unit 3 displays a scan parameter setting screen shown in FIG. 6 on a right screen.

At Step A2, the operator clicks a new patient (New Patient) on the scan parameter setting screen for the shuttle mode helical scan shown in FIG. 6.

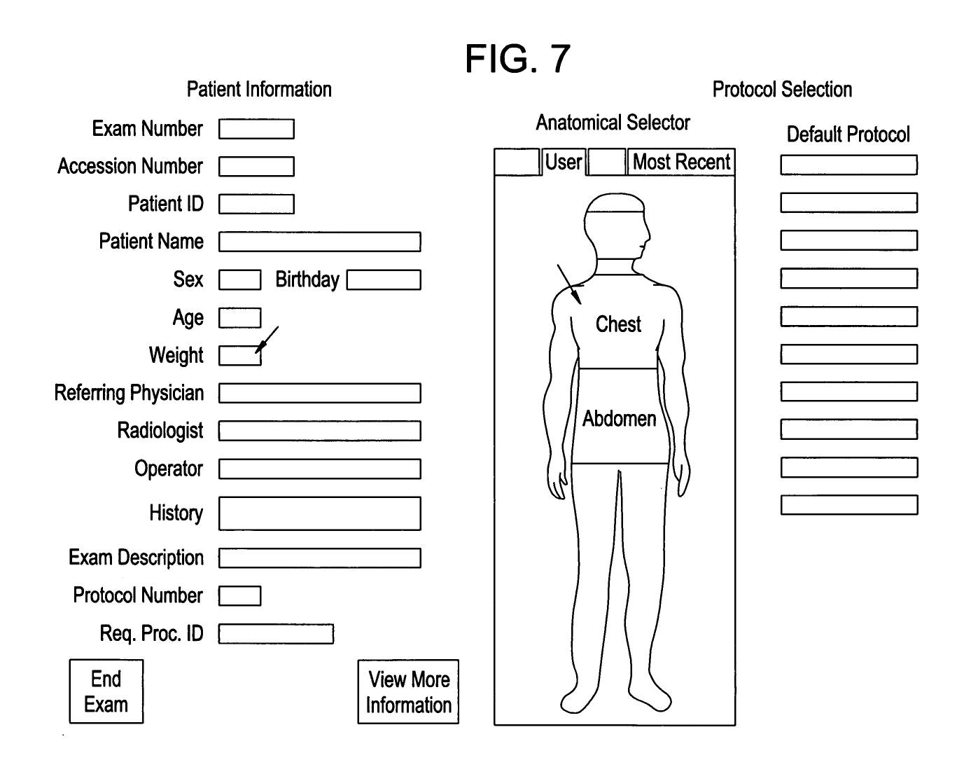

At Step A3, the central processing unit 3 displays a patient information screen (Patient Information) and a protocol selection screen (Protocol Selection) shown in FIG. 7 on the right screen.

At Step A4, the operator inputs the weight of a patient and the like on the patient information screen shown in FIG. 7.

At Step A5, the operator clicks a portion or region to be imaged on a partial selection screen (Anatomical Selector) of the protocol selection screen shown in FIG. 7. Here, the operator clicks, for example, a chest.

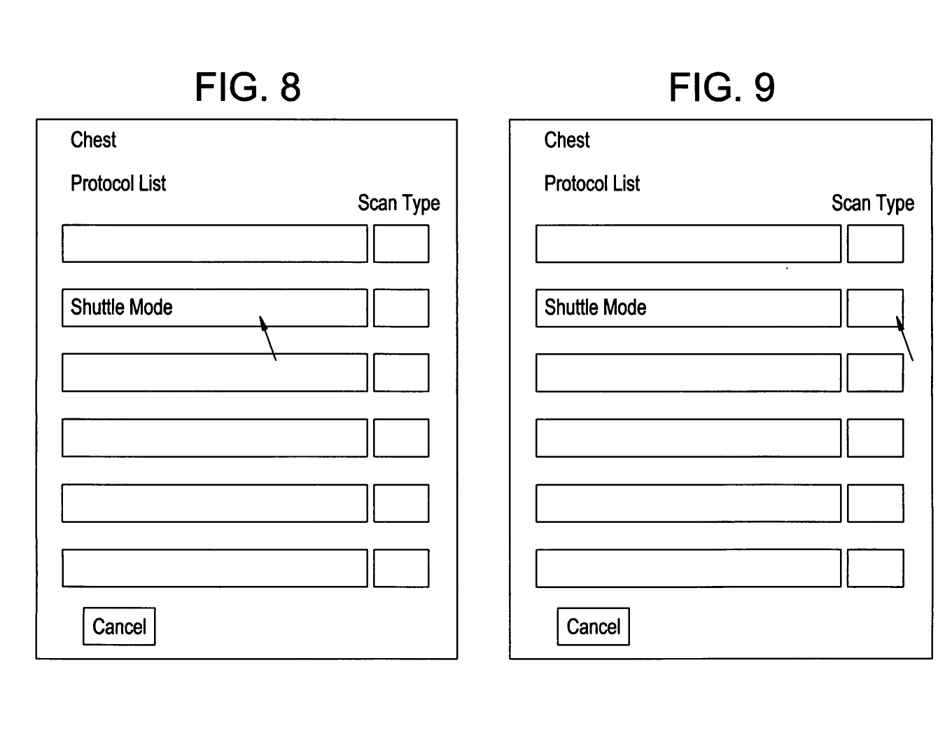

At Step A6, the central processing unit 3 pop-up displays protocol list screens (Protocol Lists) shown in FIGS. 8 and 9.

The operator clicks a desired protocol at Step A7 as shown in FIG. 8 or clicks a scan type corresponding to a desired protocol at Step A8 as shown in FIG. 9.

When the operator clicks the desired protocol, i.e., a Shuttle Mode herein at Step A7 as shown in FIG. 8, the central processing unit 3 erases the protocol list screen and proceeds to Step A21 shown in FIG. 5.

When the operator clicks the scan type corresponding to the desired protocol, i.e., the scan type associated with the shuttle mode herein at Step A8 as shown in FIG. 9, the central processing unit 3 proceeds to Step A9.

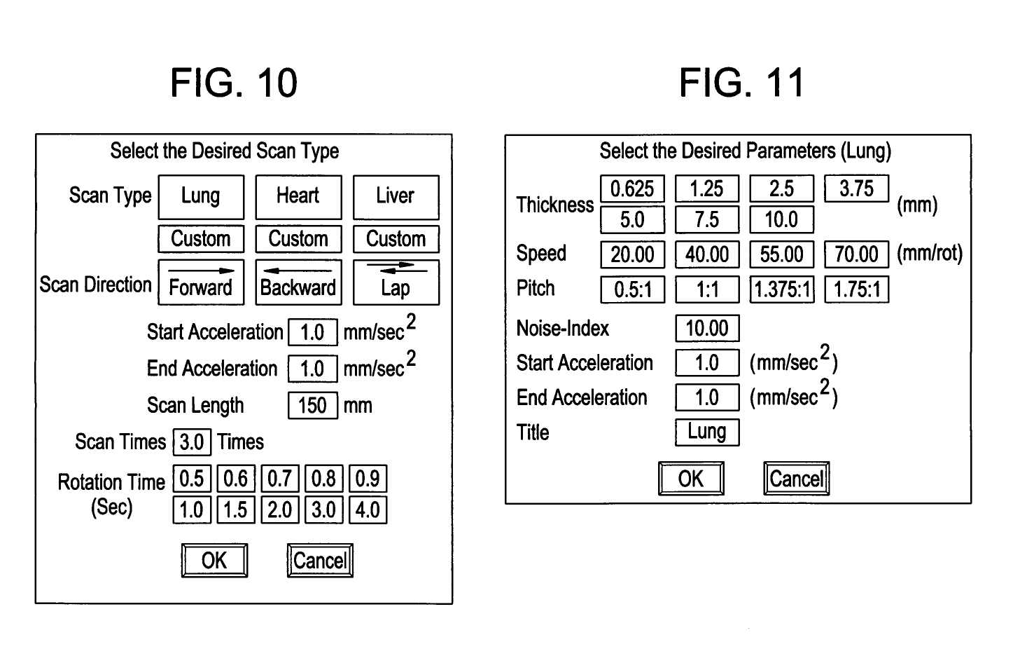

At Step A9, the central processing unit 3 pop-up displays a scan type setting screen (Select the desired Scan Type) shown in FIG. 10.

The operator selects a desired region (Lung or the like), a Scan Direction, a Rotation Time as shown at Step A10 on the scan type setting screen of FIG. 10, selects or key-inputs the values of a Start Acceleration, an End Acceleration, a Scan length and Scan Times and clicks an OK, or double-clicks a desired region (Lung or the like) as shown at Step A11.

Incidentally, any of a forward direction, a backward direction and shuttling is selected as the scan direction. As the scan times, the number of times that the forward scan, the backward scan or the shuttle scan is repeated with respect to its corresponding range, is selected and key-inputted.

When the operator performs the selection of the desired region and the like as shown at Step A10 and clicks an OK, the central processing unit 3 erases the scan type setting screen and returns to Step A6.

When the operator double-clicks a desired region, e.g., each lung as shown at Step All, the central processing unit 3 proceeds to Step A12.

At Step A12, the central processing unit 3 pop-up displays a scan parameter selection screen (Select the desired Parameters) shown in FIG. 11. In the scan parameter selection screen, default values or previous set values are selected or set as candidates for parameter values.

If the default values or previous set values selected or set on the scan parameter selection screen shown in FIG. 11 are permitted, then the operator clicks an OK at Step A13. When it is desired to change the values, the operator selects or key-inputs desired values. As to, for example, a slice thickness (Thickness), a table speed (Speed), and a helical pitch (Pitch), the operator selects their corresponding values. As to a noise index (Noise-Index), start acceleration (Start Acceleration), end acceleration (End Acceleration), and a region name (Title), the operator key-inputs their corresponding values. And the operator clicks an OK. When the operator clicks the OK, the central processing unit 3 erases the scan parameter selection screen and returns to Step A9.

Incidentally, there is a need to set the respective values in such a manner than the relationship of “table speed”/“slice-direction actually-used width of multi-row X-ray detector 24”=“helical pitch” is established. Since the “slice-direction actually-used width of multi-row X-ray detector 24”=64 rows×0.625 mm: default in the previous numerical example, the relationship of 55 (mm/rot)/40 (mm)=1.375 is established.

The noise index is a target value for a standard deviation (SD) of a pixel value of a tomographic image at the use of an auto tube current setting function (Auto mA).

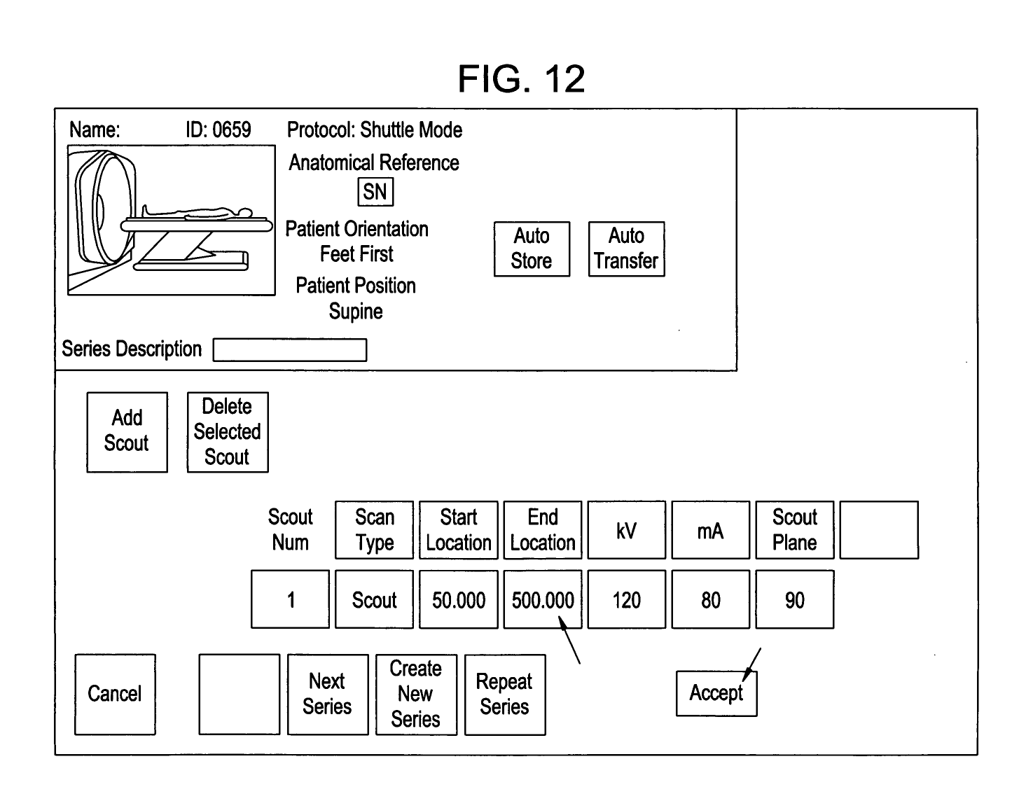

At Step A21 shown in FIG. 5, the central processing unit 3 displays a scout scan screen shown in FIG. 12 on the right screen. In the scout scan screen, default values or previous set values are selected or set as candidates for parameter values.

If the default values or previous set values selected or set on the scout scan screen of FIG. 12 are permitted, then the operator clicks an acceptance (Accept) at Step A22. When it is desired to change each value, the operator selects each desired item and key-inputs its value. When, for example, the lung is designated as a region, a general start position (Start Location) and end position (End Location) associated with the lung are set as candidates. However, the operator may key-input the value of the end location so as to contain even, for example, a liver (Liver) according to desires and click an acceptance (Accept).

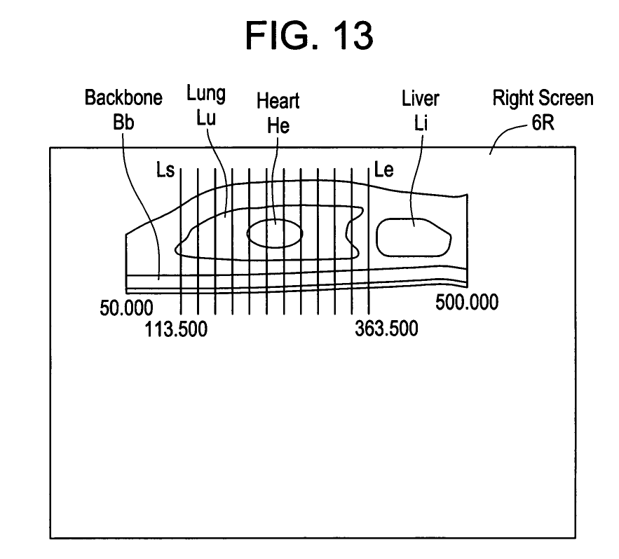

At Step A23, the central processing unit 3 executes a scout scan. That is, the X-ray tube 21 and the multi-row X-ray detector 24 are fixed so as to be opposed in the horizontal direction (Scout Plane=90), for example. Further, while the cradle 12 is being linearly moved, X rays are applied to collect scout data. Then, a scout image (X-ray penetrated image) is generated from the scout data, and the corresponding scout image is displayed on the left screen 6L of the display device 6 as shown in FIG. 13. When, for example, the lung is designated as a region with being superimposed on the scout image, a slice position or location from a general start slice location Ls associated with the lung to an end location Le associated therewith is displayed.

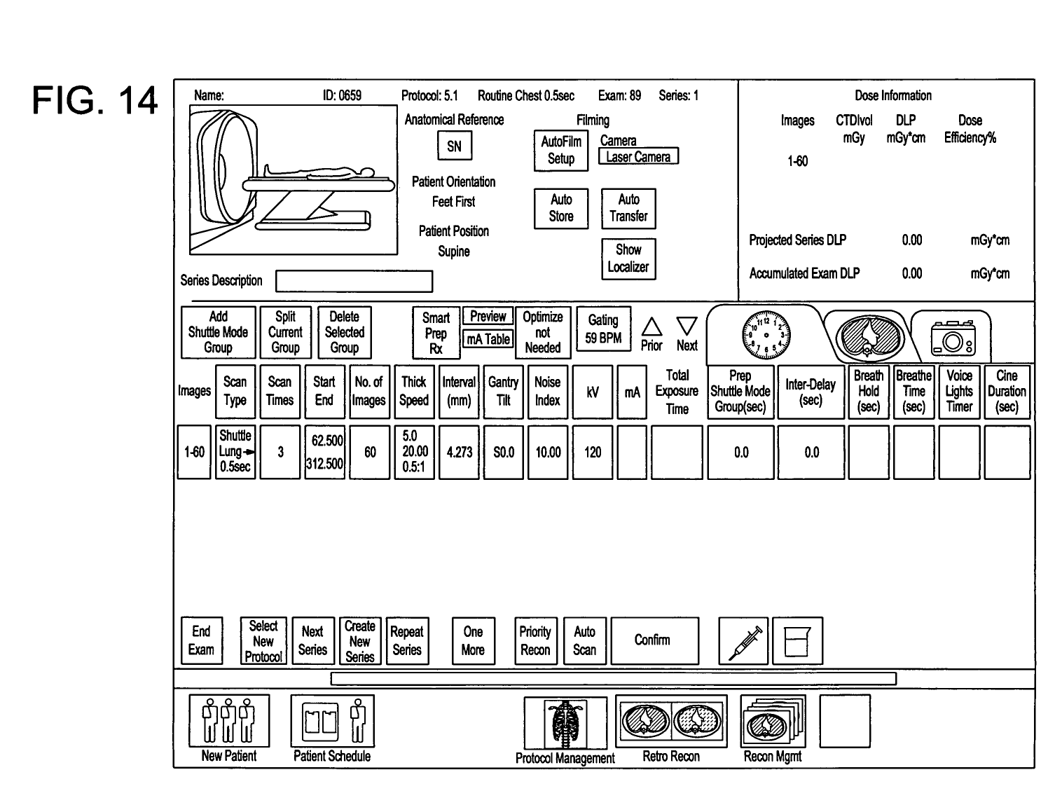

The central processing unit 3 displays a scan parameter setting screen on the right screen of the display device 6 as shown in FIG. 14. Parameters for performing a shuttle mode helical scan on the region (e.g., lung) designated by the operator are displayed on the scan parameter setting screen.

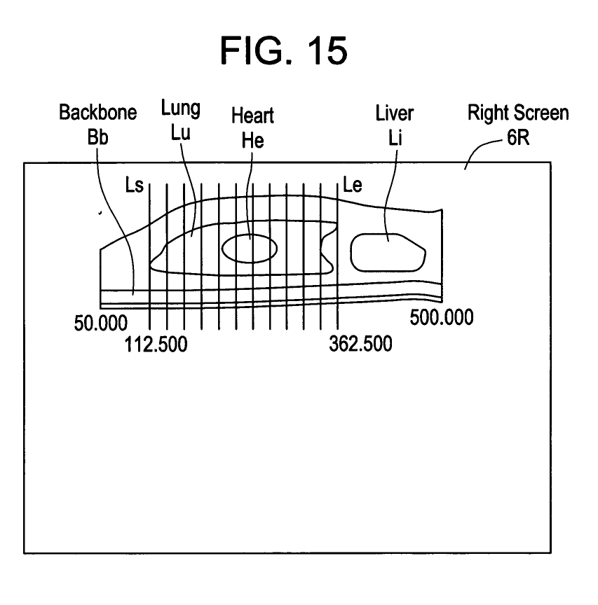

At Step A24, the operator drags & drops the display of the slice location and sets a desired slice location as shown in FIG. 15. In accordance with it, the central processing unit 3 recognizes a range from the set start location Ls to the end location Le as one range. The central processing unit 3 also recognizes one set range as one group.

At Step A25, the operator performs a change in scan parameter and/or addition of another range.

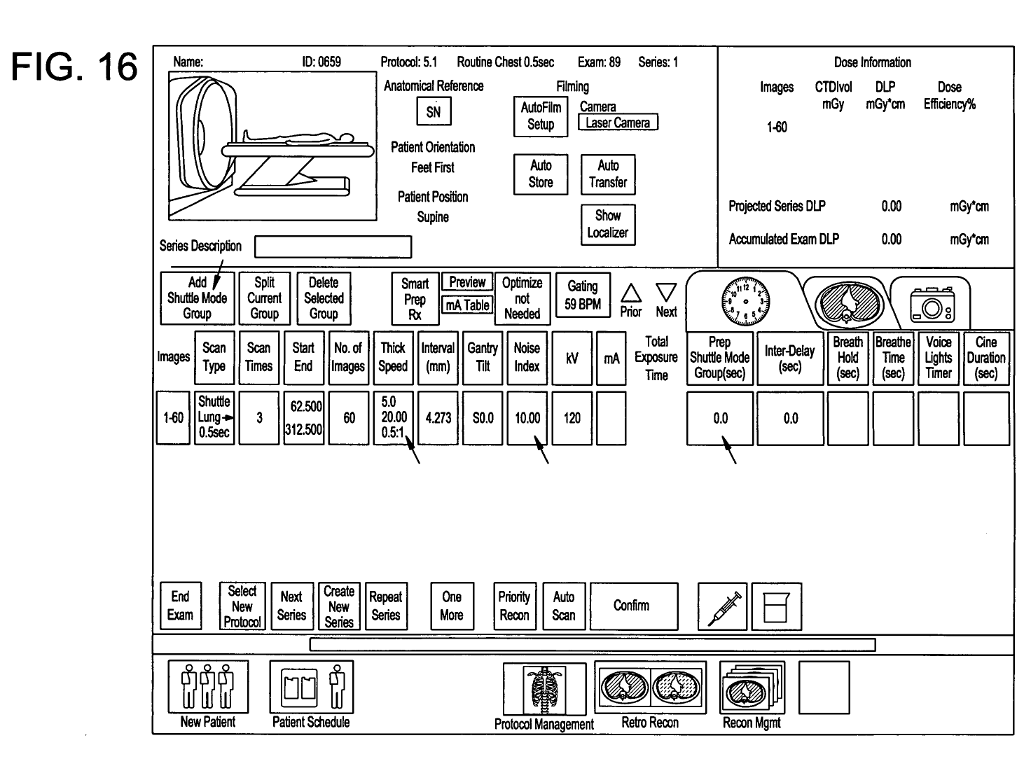

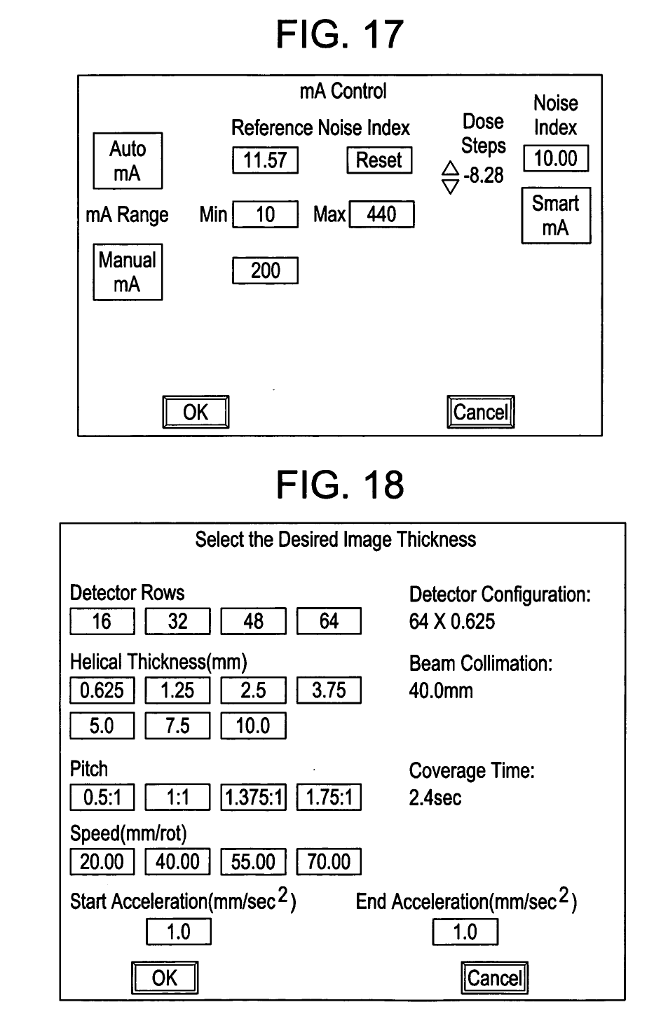

When, for example, the value of the noise index (Noise Index) is clicked, the central processing unit 3 pop-up displays a tube current setting screen (mA Control) shown in FIG. 17 on a scan parameter setting screen for a shuttle mode helical scan shown in FIG. 16, for example. The operator therefore selects, for example, an auto setting (Auto mA) on the tube current setting screen shown in FIG. 17, key-inputs the value of the noise index, e.g., “10.00” and clicks an OK. In doing so, the central processing unit 3 automatically sets a tube current, based on the noise index. And the central processing unit 3 erases the tube current setting screen shown in FIG. 17 and displays the scan parameter setting screen shown in FIG. 16.

When, for example, the value of a thickness/speed (Thick Speed) is clicked on the scan parameter setting screen for the shuttle mode helical scan shown in FIG. 16, for example, the central processing unit 3 pop-up displays a slice thickness/and the like setting screen (Select the desired Image Thickness) shown in FIG. 18. Hence, the operator sets/changes desired values.

When, for example, a group preparation time (Prep Shuttle Mode Group) is clicked on the scan parameter setting screen for the shuttle mode helical scan shown in FIG. 16, for example, the value can be key-inputted. Hence, the operator key-inputs a desired value. Incidentally, the Prep Shuttle Mode Group is a preparation time placed prior to the start of scanning of the corresponding group. Although “0.0” is set as an initial or default value, it means that the scan of the corresponding group is started immediately without providing the preparation time. If, for example, a group executed prior to a given group exists and a group preparation time for the given group is given as “0.0”, then the scan of the given group is executed following the scan of a previous group. If the group preparation time for the given group is given as “1.0”, then the scan of the given group is executed from after one-second stoppage after the previous group has been scanned.

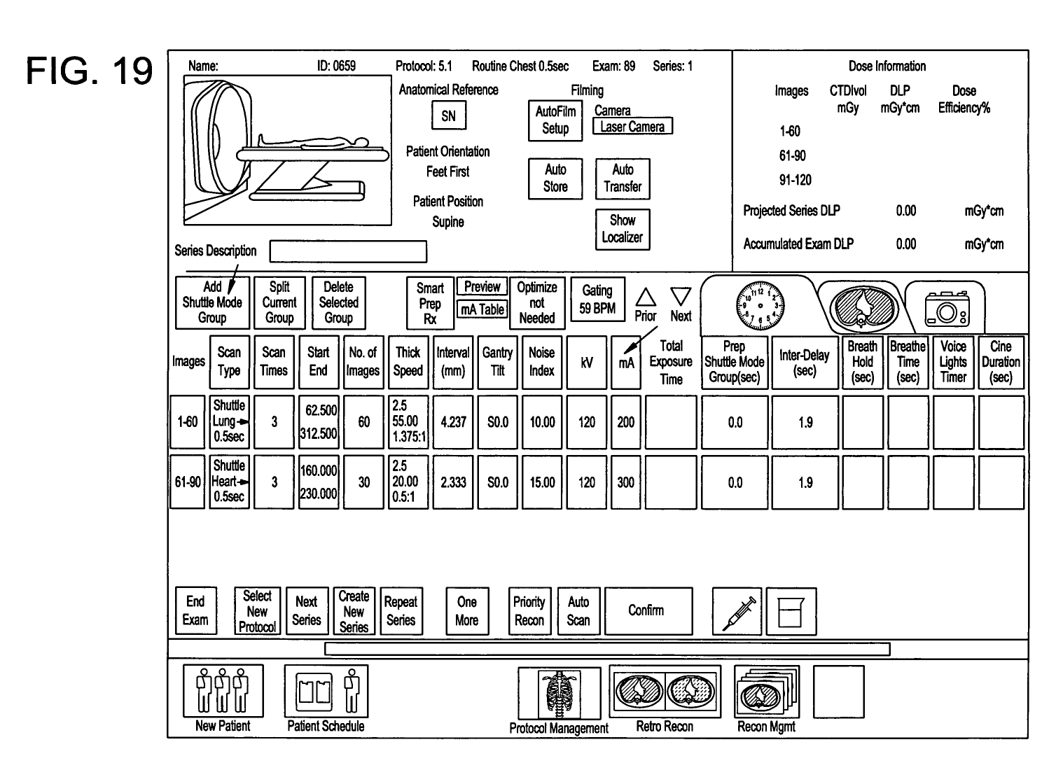

Further, when shuttle mode helical scan/group addition (Add Shuttle Mode Group) is clicked on the scan parameter setting screen for the shuttle mode helical scan shown in FIG. 16, the central processing unit 3 returns to Step A3 shown in FIG. 4. Therefore, the central processing unit 3 repeats Steps A3 to A24 and sets scan parameters for a shuttle mode helical scan corresponding to the next range as shown in FIG. 19.

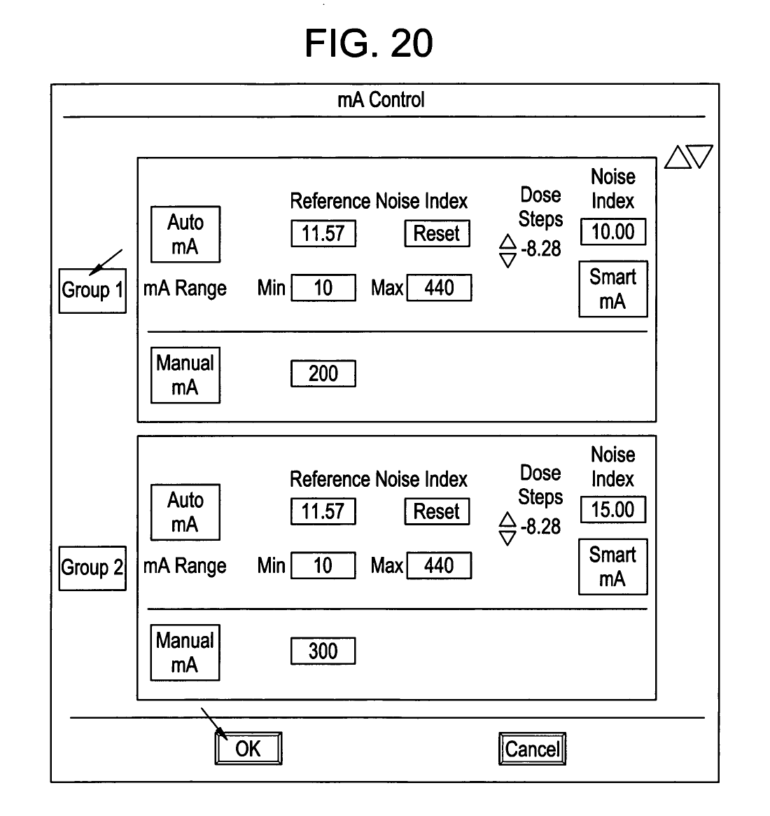

When, for example, a tube current setting button (mA) is clicked on the scan parameter setting screen for the shuttle mode helical scan shown in FIG. 19, the central processing unit 3 displays a tube current/and the like display screen (mA Control) shown in FIG. 20. Scan parameters such as tube currents set to respective ranges (groups) are displayed on the tube current/and the like display screen. When, for example, a group 1 (Group 1) is clicked here, the central processing unit 3 pop-up displays the tube current setting screen shown in FIG. 17. When OK is clicked, the central processing unit 3 returns to the scan parameter setting screen shown in FIG. 19.

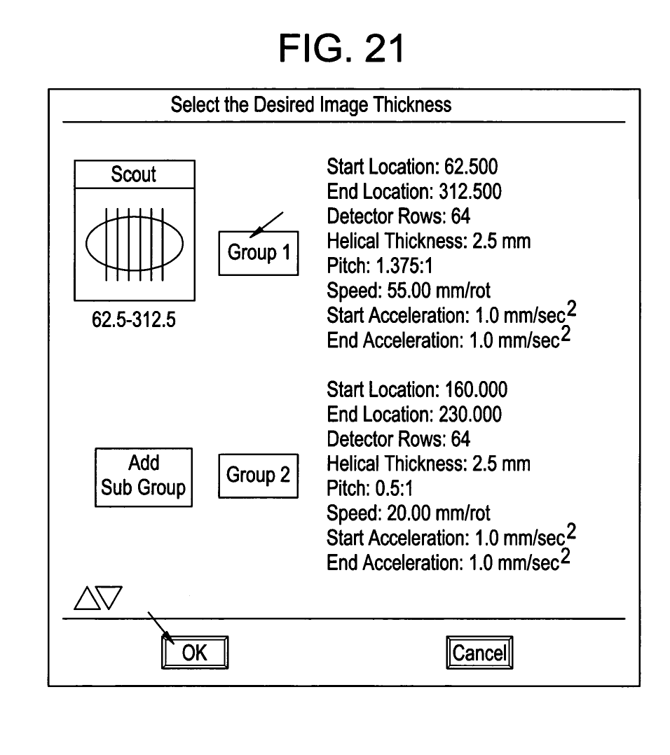

When, for example, a slice thickness/speed setting button (Thick Speed) is clicked on the scan parameter setting screen for the shuttle mode helical scan shown in FIG. 19, the central processing unit 3 displays a slice thickness/and the like display screen (Select the desired Image Thickness) shown in FIG. 21. Scan parameters such as a slice thickness, etc. set to respective ranges (groups) are displayed on the slice thickness/and the like display screen. When, for example, a group 1 (Group1) is clicked, the central processing unit 3 pop-up displays a slice thickness/and the like setting screen shown in FIG. 18. When OK is clicked, the central processing unit 3 returns to the scan parameter setting screen shown in FIG. 19.

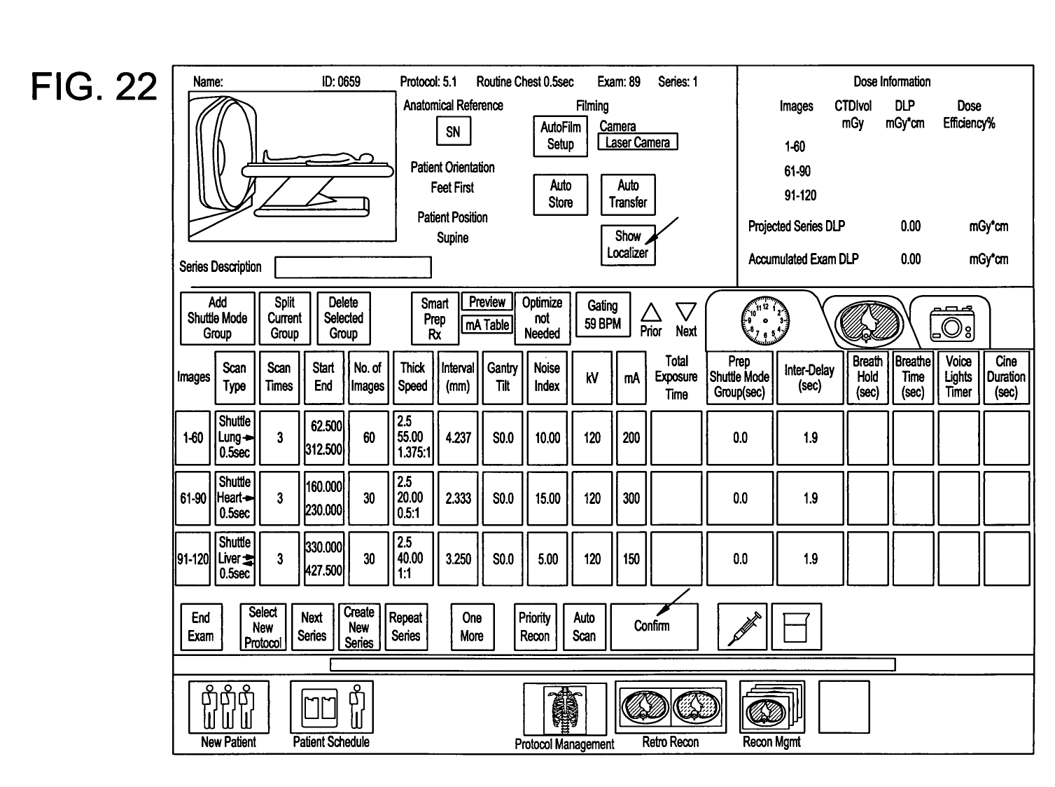

Group addition is repeated according to desires on the scan parameter setting screen for the shuttle mode helical scan shown in FIG. 19, and scan parameters for the shuttle mode helical scan associated with a plurality of ranges are set as shown in FIG. 22.

When changes in scan parameter and/or addition of another range are completed, the central processing unit 3 proceeds to Step A26 of FIG. 5 and the operator performs a parameter change graphic display, and the registration and confirmation of each series name.

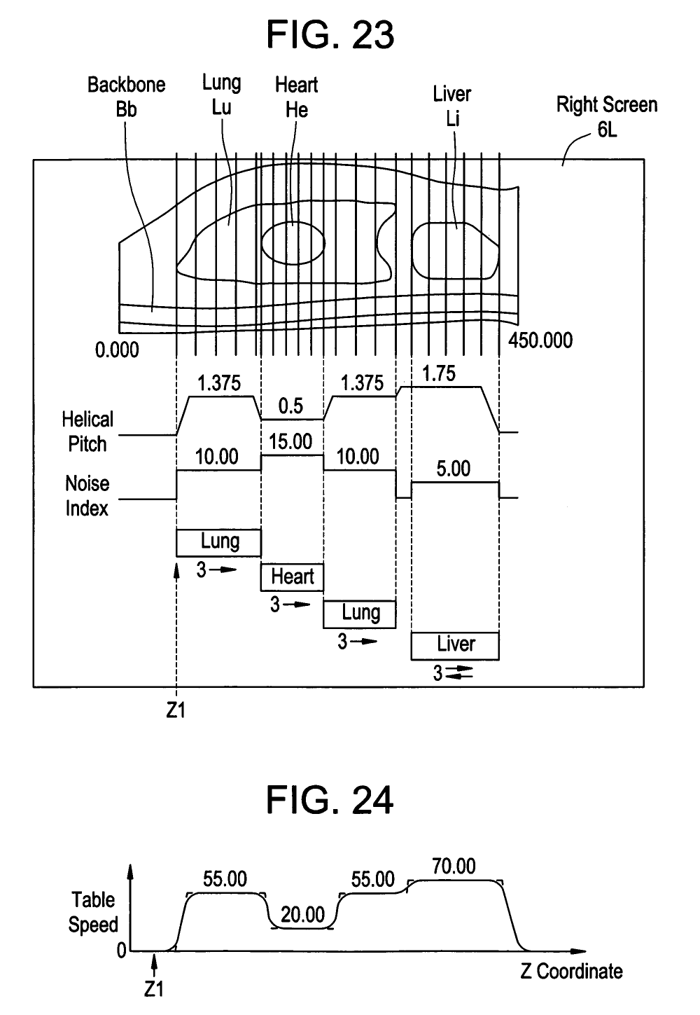

When, for example, a parameter change display (Show Localizer) is clicked on a scan parameter setting screen shown in FIG. 22, the central processing unit 3 displays a scout image and changes in main scan parameters on the left screen 6L of the display device 6 as shown in FIG. 23.

Now, the changes in scan parameters are planned according to the following rules.

(1) Acceleration/deceleration is performed within a range (the acquisition of projection data is carried out even during acceleration/deceleration).

(2) When a given range and another range partly overlap, a helical pitch (linear travel speed corresponding to one slow in speed, of both ranges) associated with one small in helical pitch, of both ranges is given a priority where a resolution priority (Resolution) is selected. When a low dose priority (Low Dose) is selected in reverse, a helical pitch (linear travel speed associated with one fast in speed, of them) associated with one large in helical pitch, of them is given a priority.

(3) Since a low dose is made between the ranges, the cradle linearly travels at a helical pitch (at a linear travel speed associated with one fast in linear travel speed, of the two) associated with one large in helical pitch, of the two.

(4) Since the low dose is made between the ranges, the application of X rays is stopped.

(5) Acceleration/deceleration is planned based on a predetermined function with set start acceleration as the base.

As a result, the changes in scan parameters become such changes in helical pitch and noise index as shown in FIG. 23.

Incidentally, although the predetermined function for the acceleration/deceleration is linear in FIG. 23, the predetermined function may be nonlinear as shown in FIG. 24.

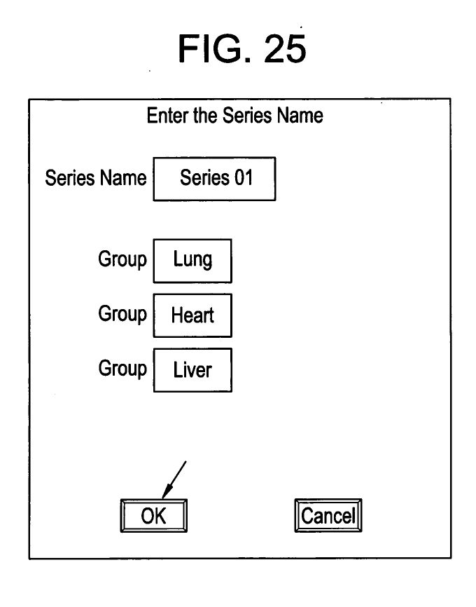

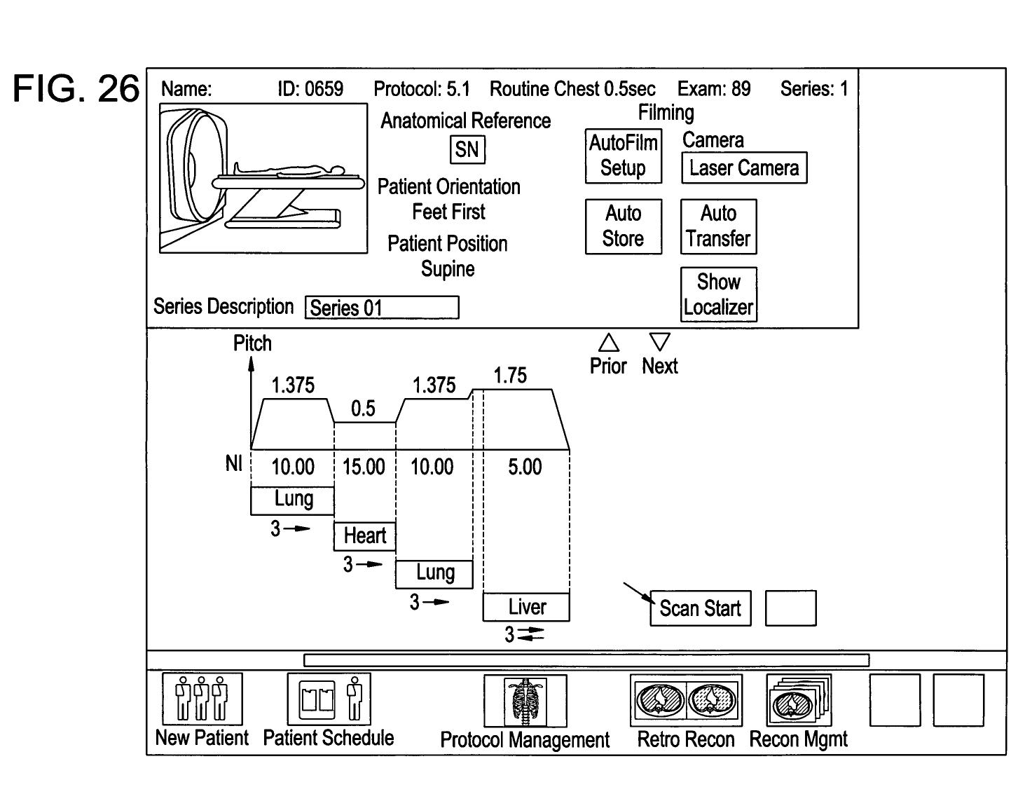

When confirmation (Confirm) is clicked on the scan parameter setting screen shown in FIG. 22, the central processing unit 3 displays a series registration screen (Enter the Series Name) shown in FIG. 25 on the right screen of the display device 6. Therefore, the operator key-inputs a series name on a series registration screen shown in FIG. 26 and clicks an OK. In doing so, the central processing unit 3 registers one or more set groups as one series and proceeds to Step A27. The registered series (chain of parameter groups) can be reused by designating and invoking the corresponding series name.

At Step A27, the central processing unit 3 displays a scan progress screen shown in FIG. 26 on the right screen of the display device 6.

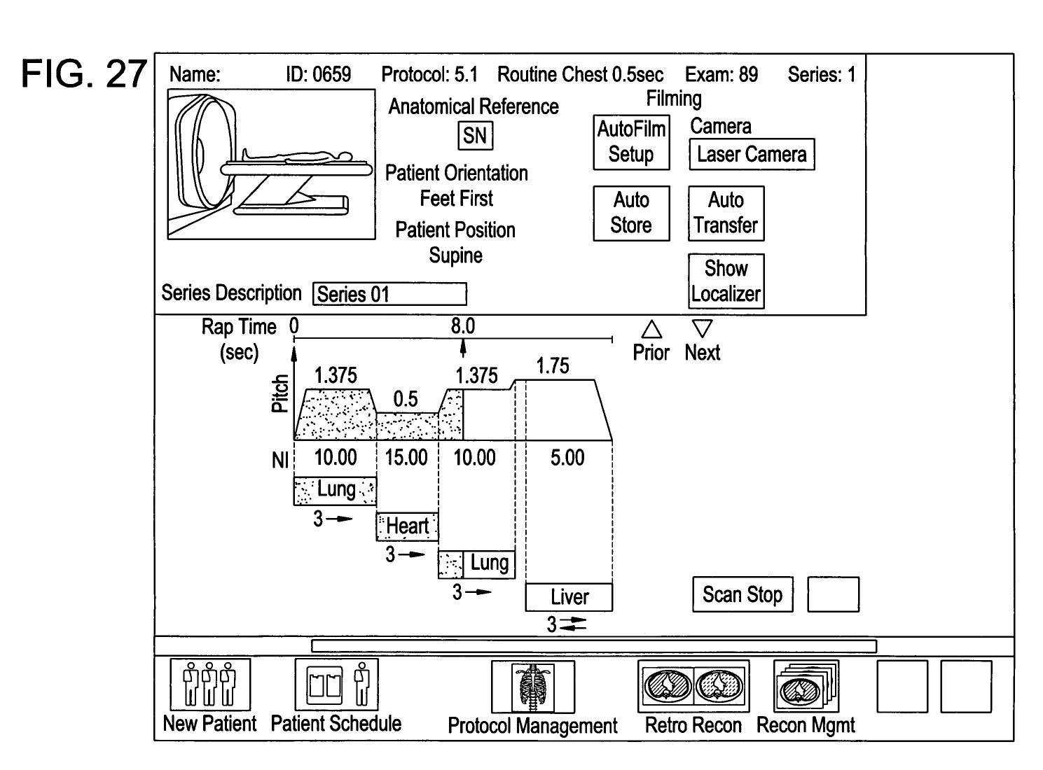

At Step A28, the operator clicks a scan start (Scan Start) on the scan progress screen of the display device 6. In doing so, the central processing unit 3 starts a data acquisition process (process 2 of FIG. 3). The central processing unit 3 displays the situation of progress of the data acquisition process on the scan progress screen as shown in FIG. 27.

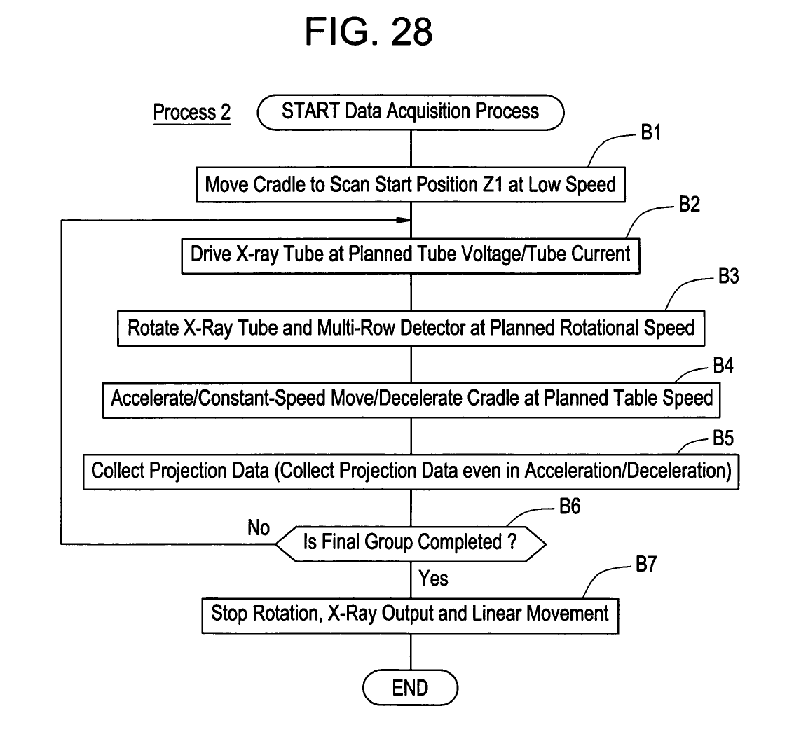

FIG. 28 is a flowchart showing the details of the data acquisition process (process 2 of FIG. 3).

At Step B1, the cradle 12 is linearly moved at a low speed from a start point Z1 shown in FIG. 23 to a position where the X-ray cone beam CB passes.

At Step B2, the X-ray tube 21 is driven by a planned tube voltage/tube current according to the present z coordinate of the cradle 12.

At Step B3, the X-ray tube 21 and the multi-row X-ray detector 24 are rotated at a planned rotational speed according to the present z coordinate of the cradle 12.

At Step B4, the cradle 12 is accelerated/constant-speed moved/decelerated at a planned table speed according to the present z coordinate of the cradle 12.

At Step B5, projection data are collected (collected even in acceleration/deceleration).

At Step B6, it is checked whether the final group has been finished. If it is found that the final group has not been finished, then Steps B2 through B5 are repeated. If it is found that the final group has been finished, then the data acquisition process proceeds to Step B7.

At Step B7, the rotation of the X-ray tube 21 and the multi-row X-ray detector 24, the output of the X rays and the linear traveling of the cradle 12 are stopped and the data acquisition process is terminated.

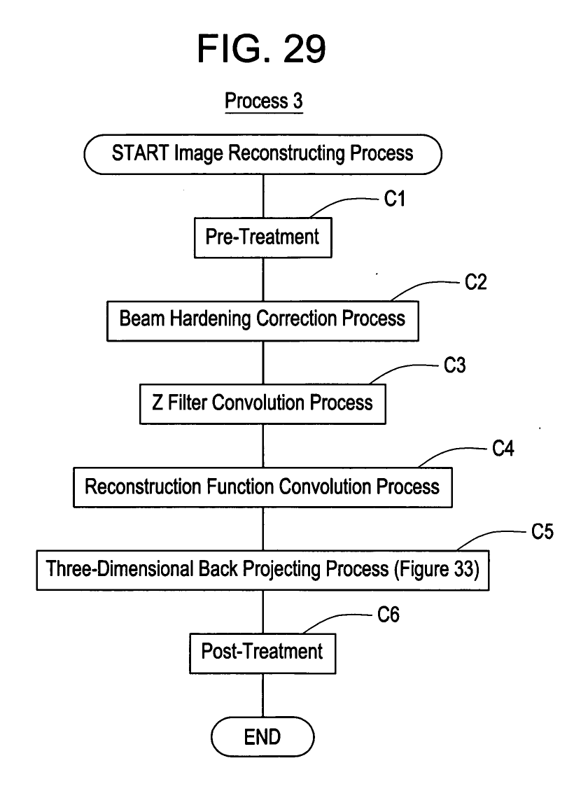

FIG. 29 is a flowchart showing the details of the image reconstructing process (process 3 of FIG. 3).

At Step C1, a pre-treatment including an offset correction, logarithmic transformation, an X-ray dose correction and a sensitivity correction is effected on projection data D0(α, z, view, j, i) expressed in a tilt angle α, a table linear traveling position z, a view angle view, a detector row number j and a channel number i to obtain projection data Din(α, z, view, j, i).

At Step C2, a beam hardening process is effected on the pre-treated projection data Din(α, z, view, j, i). The beam hardening process is expressed in the following polynomial equation, for example. In the polynomial equation, B₀, B₁ and B₂ indicate beam hardening coefficients respectively.

Dout(α, z, view, j, i)=Din(α, z, view, j, i)×(B₀(j, i)+B₁(j, i)×Din(α, z, view, j, i)+B₂(j, i)×Din(α, z, view, j, i)²)

Since beam hardening corrections independent of one another every respective rows of the detector can be performed at this time, differences in characteristic every respective detector rows can be corrected if tube voltages of respective data acquisition systems differ according to imaging conditions.

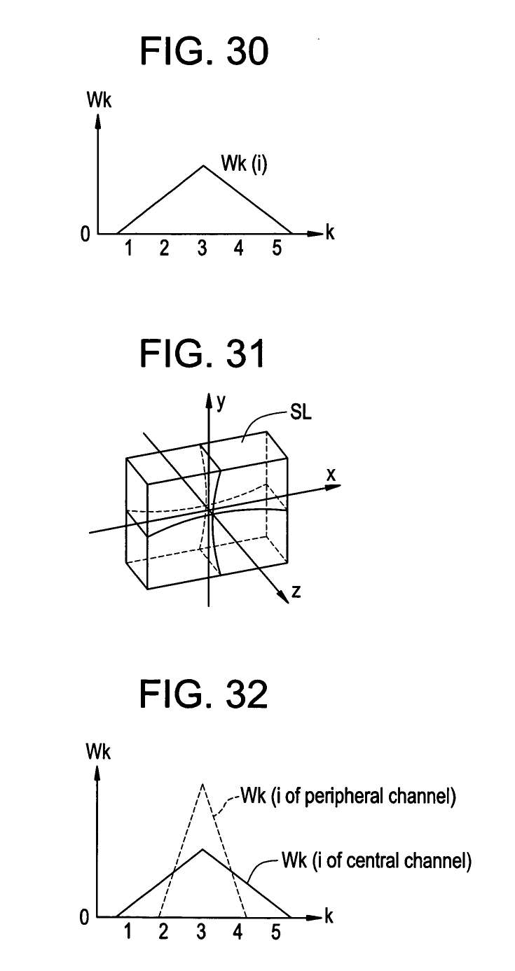

At Step C3, a Z filter convolution process for performing multiplication of a filter in a z direction (row direction) is effected on the projection data Dout(α, z, view, j, i) subjected to the beam hardening correction. That is, the projection data Dout(α, z, view, j, i) subjected to the beam hardening correction are multiplied by, for example, such row-direction filter coefficients Wk(i) as shown in FIG. 30 in the row direction to determine projection data Dcor(α, z, view, j, i).

$$Dcor ( α , z , view , j , i ) = {\sum }_{k = 1}^{5} ( Dout ( α , z , view , j + k - 3 , i ) \times Wk ( i ) )$$

$$whe re$$

$${\sum }_{k = 1}^{5} ( Wk ( i ) ) = 1$$

$$Dout ( α , z , view , - 1 , i ) = Dout ( α , z , view , 0 , i ) = Dout ( α , z , view , 1 , i )$$

$$Dout ( α , z , view , J + 1 , i ) = Dout ( α , z , view , J + 2 , i ) = Dout ( α , z , view , J , i )$$

A slice thickness can be controlled by a row-direction filter coefficient Wk(i).

In a slice SL as shown in FIG. 31, its peripheral slice thickness generally becomes thick as compared with a reconstruction center.

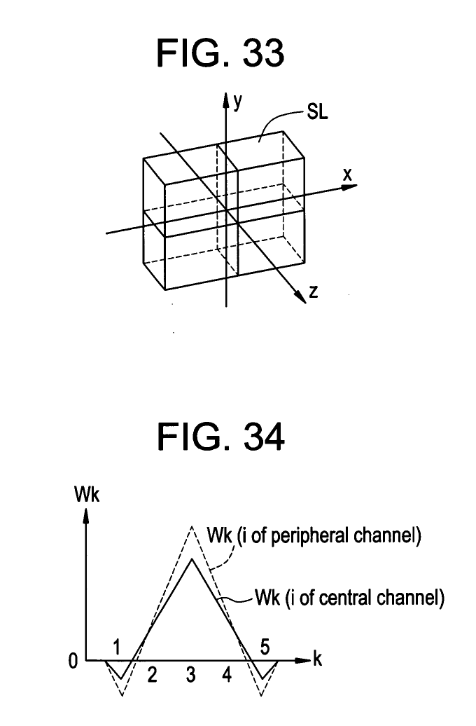

Therefore, as shown in FIG. 32, a row-direction filter coefficient Wk (i of central channel) at which the central channel is broadly changed in width, is used, and a row-direction filter coefficient Wk (i of peripheral channel) at which the peripheral channel is narrowly changed in width, is used. Thus, it is possible to set a slice SL having a slice thickness close to uniformity even at the reconstruction center and the periphery as shown in FIG. 33.

Both Archfacts and noise are greatly improved when the slick thickness is slightly made thick by the row-direction filter coefficients Wk(i). Consequently, the manner of an improvement in archfact and the manner of an improvement in noise can also be controlled. That is, the quality of a three-dimensional image reconstructed tomographic image can be controlled.

By setting the row-direction filter coefficients Wk(i) to de-convolution filters as shown in FIG. 34, a tomographic image having a thin slice thickness can also be realized.

Referring back to FIG. 29, a reconstruction function convolution process is carried out at Step C4. That is, a Fourier transform is performed on projection data, each of which is followed by being multiplied by a reconstruction function to perform an inverse Fourier transform thereof. Assuming that the projection data subsequent to the reconstruction function convolution process is defined as Dr(α, z, view, j, i), the reconstruction function is defined as Kernel(j), and convolution computation is expressed in *, the reconstruction function convolution process is expressed as follows:
Dr(α,z,view,j,i)=Dcor(α,z,view,j,i)*(Kernel(j)

Since the independent reconstruction function convolution process can be carried out using the reconstruction function Kernel(j) independent for each row of the detector, differences in noise and resolution characteristics every respective detector rows can be corrected.

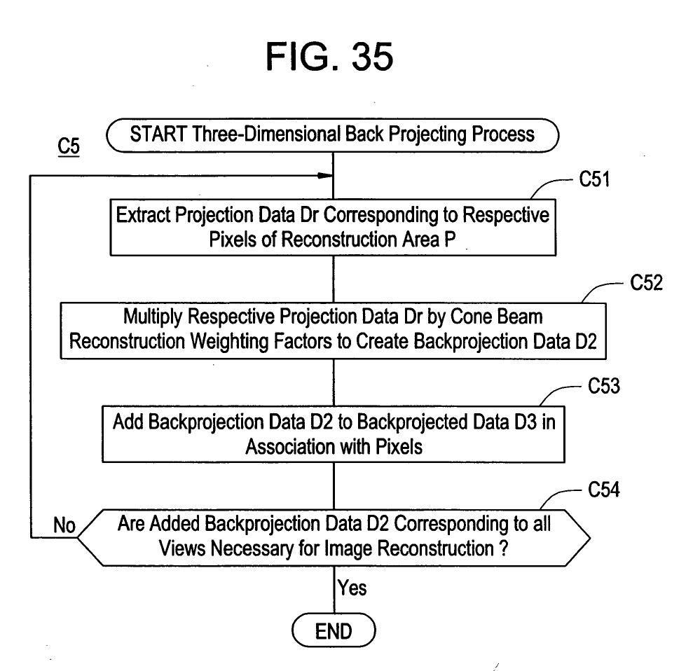

At Step C5, a three-dimensional back projecting process is effected on the projection data Dr(α, z, view, j, i) to determine backprojected data D3(x, y). The three-dimensional back projecting process will be explained later with reference to FIG. 35.

At Step C6, post-treatment such as an image filter convolution process, a CT value converting process, etc. are effected on the backprojected data D3(x, y) to obtain a tomographic image.

Assuming that data subsequent to the image filter convolution process is defined as D4(x, y), a detector row number corresponding to a central pixel of the tomographic image is defined as j, and an image filter is defined as Filter(j), the image filter convolution process is expressed as follows:
D4(x,y)=D3(x,y)*Filter(j)

That is, since the image filter convolution process independent for each slice position of the tomographic image can be carried out, differences in noise and resolution characteristics every slice positions can be corrected.

FIG. 35 is a flowchart showing the details of the three-dimensional back projecting process (Step C5 of FIG. 29).

At Step C51, attention is paid to one of all views (i.e., views corresponding to 360° or views corresponding to “180°+fan angle”) necessary for image reconstruction of the tomographic image. Projection data Dr corresponding to respective pixels of a reconstruction area R are extracted.

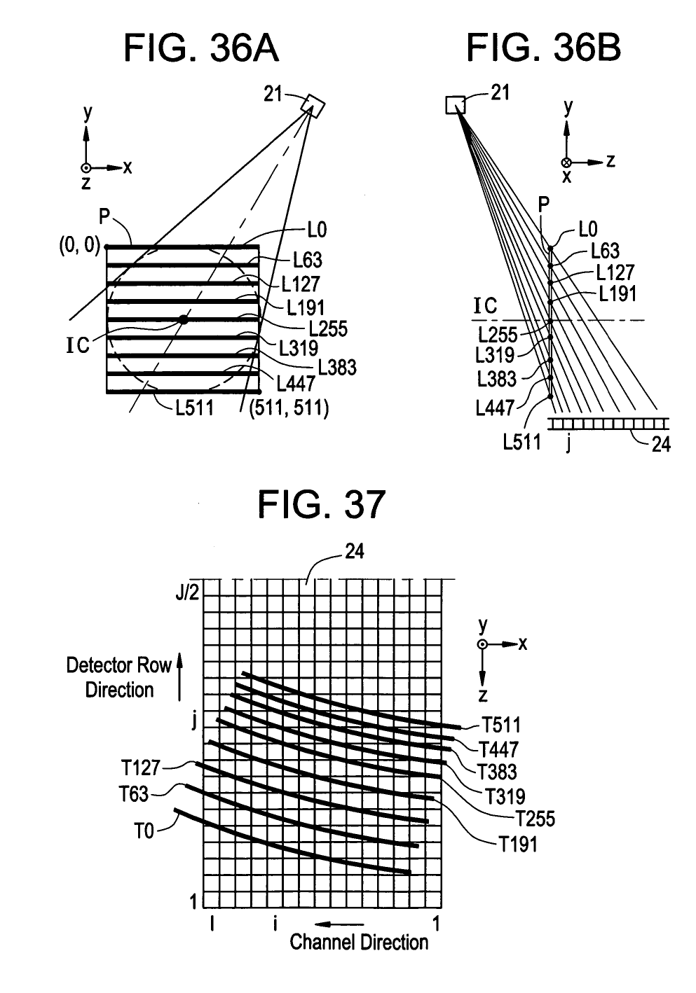

As shown in FIG. 36, a square reconstruction area P of 512×512 pixels, which is parallel to an xy plane, is considered. A pixel row L0 parallel to an x axis of y=0, a pixel row L63 of y=63, a pixel row L127 of y=127, a pixel row L191 of y=191, a pixel row L255 of y=255, a pixel row L319 of y=319, a pixel row L383 of y=383, a pixel row L447 of y=447, and a pixel row L511 of y=511 are taken by way of example. Thus, if projection data D0 on lines T0 through T511 shown in FIG. 37, which are obtained by projecting these pixel rows L0 through L511 onto the surface of the multi-row X-ray detector 24 in an X-ray penetration direction, are extracted, they result in projection data Dr corresponding to the pixel rows L0 through L511.

The X-ray penetration direction is determined depending upon geometrical positions of an X-ray focal point of the X-ray tube 21, the respective pixels and the multi-row X-ray detector 24. Since, however, the z coordinates of the projection data D0(α, z, view, j, i) are already known, the X-ray penetration direction can be accurately determined even in the case of the projection data D0(α, z, view, j, i) being in acceleration/deceleration.

When part of each line falls outside the surface of the multi-row X-ray detector 24 as in the case of, for example, the line T0 obtained by projecting, for example, the pixel row L0 onto the surface of the multi-row X-ray detector 24 in the X-ray penetration direction, its corresponding projection data Dr is set to “0”.

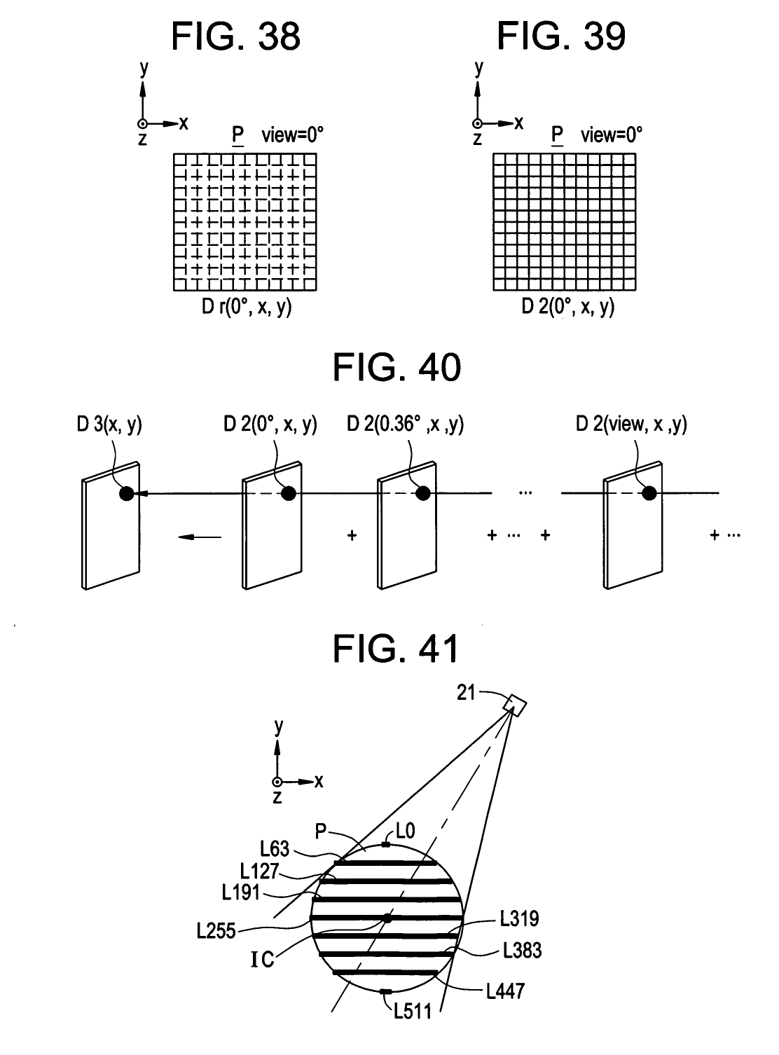

Thus, projection data Dr(view, x, y) corresponding to the respective pixels of the reconstruction area P can be extracted as shown in FIG. 38.

Referring back to FIG. 35, at Step C52, the projection data Dr(view, x, y) are multiplied by cone beam reconstruction weighting factors or coefficients to create projection data D2(view, x, y) shown in FIG. 39.

Now, the cone beam reconstruction weighting factors are as follows:

Assuming that in the case of a fan beam image reconstruction, the angle which a straight line connecting the focal point of the X-ray tube 21 and a pixel g(x, y) on the reconstruction area P (on the xy plane) forms with a central axis Bc of an X-ray beam at view=βa, is taken as γ, and an opposite view thereof is taken as view=βb, βb is expressed as follows:
βb=βa+180°−2γ

Assuming that the angles which the X-ray beam passing through the pixel g(x, y) on the reconstruction area P and its opposite X-ray beam form with the reconstruction area P, are taken as αa and αb, the corresponding backprojected data are multiplied by cone beam reconstruction weighting factors ωa and cob dependent on these angles αa and αb and then added together to determine backprojected data D2(0, x, y).
D2(0,x,y)=ωa·D2(0,x,y)_{—}a+ωb·D2(0,x,y)_{—}b

where D2(0, x, y)_a: projection data at a view βa, and D2(0, x, y)_b: projection data at a view βb.

Incidentally, the sum of the cone beam reconstruction weighting factors ωa and ωb for the X-ray beam and its opposite X-ray beam is expressed in ωa+ωb=1.

Multiplying the data by the cone beam reconstruction weighting factors ωa and ωb and adding them together as described above makes it possible to reduce cone angle archfacts.

As the cone beam reconstruction weighting factors ωa and ωb, for example, ones determined from the following equations can be used.

When a fan beam angle is assumed to be γmax with f( ) as a function, the following are given:
ga=f(π+γmax−|βa|,|tan(αa)|)
gb=f(π+γmax−|βb|,|tan(αb)|)
xa=2·ga^{q}/(ga^{q}+gb^{q})
xb=2·gb^{q}/(ga^{q}+gb^{q})
ωa=xa²·(3−2xa)
ωb=xb²·(3−2xb)

wherein, for example, f( )=max( ): function that takes a large value, and q=1.

In the case of the fan beam image reconstruction, each pixel on the reconstruction area P is further multiplied by a distance coefficient. When the distance from the focal point of the X-ray tube 21 to each of a detector row j and a channel i of the multi-row X-ray detector 24 corresponding to the projection data Dr is assumed to be r0, and the distance from the focal point of the X-ray tube 21 to the corresponding pixel on the reconstruction area P corresponding to the projection data Dr, is assumed to be r1, the distance coefficient is expressed in (r1/r0)².

A parallel beam image reconstruction is similar to the fan beam image reconstruction if βb=βa+180°. Incidentally, fan-parallel conversion corresponding to conversion processing for a parallel beam is performed in advance before and after the pre-treatment (C1 of FIG. 29), before and after the beam hardening correction process (C2 of FIG. 29) and before and after the Z filter convolution process (C3 of FIG. 29).

At Step C53, as shown in FIG. 40, projection data D2(view, x, y) are added to pre-cleared backprojected data D3(x, y) in association with pixels.

At Step C54, Steps S61 through S63 are repeated with respect to all views (i.e., views corresponding to 360° or views corresponding to “180°+fan angle”) necessary for the image reconstruction of the tomographic image, to obtain the backprojected data D3(x, y) as shown in FIG. 40.

Incidentally, the reconstruction area P may be configured as a circular area as shown in FIG. 41.

According to the X-ray CT apparatus 100 of the embodiment 1, scan parameters such as a helical pitch and noise index for a shuttle mode helical scan can efficiently be set through easy-to-understand user interfaces (scan parameter setting screen and the like). A tomographic image having sufficient image quality can be obtained by the optimum dose under the optimum imaging conditions every regions or organs of a subject.

Incidentally, the image reconstructing method may be a three-dimensional image reconstructing method based on the conventionally known Feldkamp method. Further, a three-dimensional image reconstructing method proposed in each of Japanese Unexamined Patent Publication Nos. 2003-334188, 2004-41675, 2004-41674, 2004-73360, 2003-159244 and 2004-41675 may be used.

Although the number of groups is three in the embodiment 1, an example illustrative of a larger number of groups or an example illustrative of a smaller number of groups is also similar to the embodiment 1.

Although the embodiment 1 has not explained the settings of an imaging or photographic visual-field size, a reconstruction function, an image filter and the like, image quality and exposure set for each region or organ of a subject can be optimized by setting the above every groups in a manner similar to the embodiment 1.

Many widely different embodiments of the invention may be configured without departing from the spirit and the scope of the present invention. It should be understood that the present invention is not limited to the specific embodiments described in the specification, except as defined in the appended claims.

1. A scan parameter setting method for a shuttle mode helical scan, comprising the steps of:
displaying a scout image of a subject;
allowing an operator to designate at least one range in a body-axis direction, of the scout image; and
allowing the operator to graphically input or key-input and set a helical pitch for the shuttle mode helical scan in association with the range.

2. A scan parameter setting method for a shuttle mode helical scan, comprising the steps of:
displaying a scout image of a subject;
allowing an operator to designate a plurality of ranges in a body-axis direction, of the scout image; and
allowing the operator to graphically input or key-input and set a noise index for the shuttle mode helical scan in association with each of the ranges.

3. The scan parameter setting method according to claim 1, further including a step for allowing the operator to graphically input or key-input and set a noise index for the shuttle mode helical scan in association with the range.

4. The scan parameter setting method according to claim 1, further including a step for allowing the operator to graphically input or key-input and set at least one of a tube voltage and a tube current for the shuttle mode helical scan in association with said each range.

5. The scan parameter setting method according to claim 1, further including a step for allowing the operator to set at least one of a slice thickness, a detector row number, a table speed, the number of tomographic images, a tomographic image interval and table acceleration for the shuttle mode helical scan in association with said each range.

6. The scan parameter setting method according to claim 1, further including a step for defining said one range as one group and setting one series comprising one or more groups.

7. The scan parameter setting method according to claim 2, further including a step for defining said one range as one group and setting one series comprising one or more groups.

8. The scan parameter setting method according to claim 3, further including a step for defining said one range as one group and setting one series comprising one or more groups.

9. An X-ray CT apparatus comprising:
an X-ray tube;
a detector;
a helical scan device for rotating at least one of the X-ray tube and the detector about a target to be imaged and collecting data while both are being moved linearly relative to the target to be imaged;
a scan parameter setting device for allowing an operator to set scan parameters for a helical scan; and
an image reconstructing device for reconstructing an image, based on the collected data,
wherein the scan parameter setting device displays a scout image of a subject and when the operator designates at least one range in a body-axis direction, of the scout image and graphically inputs or key-inputs a helical pitch for the shuttle mode helical scan in association with the range, the scan parameter setting device sets the inputted helical pitch as a scan parameter for the shuttle mode helical scan, corresponding to the range.

10. An X-ray CT apparatus comprising:
an X-ray tube:
a detector;
a helical scan device for rotating at least one of the X-ray tube and the detector about a target to be imaged and collecting data while both are being moved linearly relative to the target to be imaged;
a scan parameter setting device for allowing an operator to set parameters for a helical scan; and
an image reconstructing device for reconstructing an image, based on the collected data,
wherein the scan parameter setting device displays a scout image of a subject and when the operator designates at least one range in a body-axis direction, of the scout image and graphically inputs or key-inputs a noise index for the shuttle mode helical scan in association with the range, the scan parameter setting device sets the inputted noise index as a scan parameter for the shuttle mode helical scan, corresponding to the range.

11. The X-ray CT apparatus according to claim 9, wherein when the operator graphically inputs or key-inputs a noise index for the shuttle mode helical scan in association with the range, the parameter setting device sets the inputted noise index as a scan parameter for the shuttle mode helical scan, corresponding to the range.

12. The X-ray CT apparatus according to claim 9, wherein when the operator graphically inputs or key-inputs at least one of a tube voltage and a tube current for the shuttle mode helical scan in association with the range, the parameter setting device sets said at least one as a scan parameter for the shuttle mode helical scan, corresponding to the range.

13. The X-ray CT apparatus according to claim 9, wherein when the operator inputs at least one of a slice thickness, a detector row number, a table speed, the number of tomographic images, a tomographic image interval, and table acceleration for the shuttle mode helical scan in association with said each range, the parameter setting device sets said at least one as a scan parameter for the shuttle mode helical scan, corresponding to the range.

14. The X-ray CT apparatus according to claim 9, wherein the parameter setting device is capable of setting one series comprising one or more groups with said one range as one group, and when execution of one series is instructed, the helical scan device continuously executes a shuttle mode helical scan for a group which belongs to the corresponding series.

15. The X-ray CT apparatus according to claim 10, wherein the parameter setting device is capable of setting one series comprising one or more groups with said one range as one group, and when execution of one series is instructed, the helical scan device continuously executes a shuttle mode helical scan for a group which belongs to the corresponding series.

16. The X-ray CT apparatus according to claim 11, wherein the parameter setting device is capable of setting one series comprising one or more groups with said one range as one group, and when execution of one series is instructed, the helical scan device continuously executes a shuttle mode helical scan for a group which belongs to the corresponding series.

17. The X-ray CT apparatus according to claim 15, wherein the parameter setting device is capable of setting one series comprising one or more groups with said one range as one group, and when execution of one series is instructed, the helical scan device continuously executes a shuttle mode helical scan for a group which belongs to the corresponding series.

18. The X-ray CT apparatus according to claim 9, wherein the parameter setting device sets said one range in association with one organ or region.

19. The X-ray CT apparatus according to claim 9, wherein the parameter setting device automatically sets a default value of at least one scan parameter or a previous set value as a candidate for a set value with respect to said designated one range.

20. The X-ray CT apparatus according to claim 9, wherein the helical scan device performs a shuttle mode variable pitch helical scan or a shuttle mode variable speed helical scan for collecting data even at the time of a start of linear movement and an end thereof and even in acceleration or deceleration at midstream thereof.
